(19) United States
(12) Patent Application Publication
(10) Pub. No.: US 2026/0347362 A1
(43) Pub. Date: Nov. 5, 2026

(54) PHTHALAZINE DERIVATIVES AS ANTICANCER COMPOUNDS AND METHOD OF PREPARATION THEREOF

(71) Applicant: Imam Abdulrahman Bin Faisal University, Dammam (SA)

(72) Inventors: Samar A. ABUBSHAIT, Dammam (SA); Haya A. ABUBSHAIT, Dammam (SA); Rasha J. ALMALIH, Dammam (SA)

(73) Assignee: Imam Abdulrahman Bin Faisal University, Dammam (SA)

(21) Appl. No.: 19/184,725

(22) Filed: Apr. 21, 2025

(51) Int. Cl. C07D 487/04; A61K 31/502; A61K 31/5025; A61P 35/00; C07D 237/34; C07D 403/04; C07D 409/12

(52) U.S. Cl. CPC C07D 487/04; A61K 31/502; A61K 31/5025; A61P 35/00; C07D 237/34; C07D 403/04; C07D 409/12

(57) ABSTRACT

A method of synthesizing a phthalazine compound by microwave irradiation includes reacting phosphorus oxychloride, phosphorus pentachloride, and 4-(4-isopropyl-phenyl)-2H-phthalazin-1-one in an organic solvent to obtain a reaction mixture consisting of 1-chloro-4-(4-isopropyl-phenyl)-phthalazine, and mixing at least one selected from the group consisting of a hydrazine, an amine, an acetone, a dinitrile, a dicarbonyl and a semicarbazide with the reaction mixture obtain a final mixture, then microwave irradiating the final mixture at a power of 250 to 450 watt (W) for 5 to 30 minutes (min) to form the phthalazine compound.

### STATEMENT OF PRIOR DISCLOSURE BY AN INVENTOR

Aspects of the present disclosure are described in Abubshait, S. A., Almalih, R. J., Abubshait, H. A., Gomaa, M. S., & Abdalla, A. N. (2024), “Microwave-assisted synthesis, characterization, and molecular docking studies of new chlorophthalazine derivatives as anticancer activity,” Arab Journal of Basic and Applied Sciences, 31(1), 454-469, which is incorporated herein by reference in its entirety.

### STATEMENT OF ACKNOWLEDGEMENT

Support provided by the College of Science and the Basic and Applied Scientific Research Center at Imam Abdulrahman Bin Faisal University Dammam, Saudi Arabia is gratefully acknowledged.

### BACKGROUND

### Technical Field

The present disclosure is directed to phthalazine derivatives, compositions containing the phthalazine derivatives, methods of synthesizing a phthalazine compound, and methods of using the phthalazine derivatives for treating cancer.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Phthalazine derivatives are a significant class of heterocyclic compounds that have attracted considerable attention due to their wide range of biological activities and pharmacological properties. These organic compounds contain ring systems made up of carbon atoms and one or more heteroatoms, and they have demonstrated various medicinal and pharmaceutical uses. Phthalazine derivatives have shown great potential for therapeutic development, especially in antibacterial, antifungal, anti-inflammatory, antiviral, and antitumor applications. Notably, their anticancer properties have been highlighted in numerous studies, with hybrid molecules and analogues exhibiting strong antiproliferative activities against various cancer cell lines. Some derivatives have shown significant effects by targeting molecular pathways involved in cell death and tumor growth, including the inhibition of VEGFR-2 and caspase-8, and the suppression of key markers like NF-κB P65 and TNF-α.

In the context of anticancer research, phthalazine derivatives have shown promising activity against various cancer cell lines, including those associated with breast and liver cancers. These compounds, such as phthalazinone derivatives, have been identified as potent inhibitors of poly (ADP-Ribose) polymerase 1 (PARP-1), a key enzyme involved in DNA repair. The development of phthalazine-based molecules has further been explored to improve the selectivity and potency of these compounds, with the potential for enhanced therapeutic outcomes in cancer treatment. Recent efforts have focused on optimizing these derivatives by incorporating different functional groups and exploring their molecular mechanisms through docking studies.

The goal of these investigations is to evaluate the anticancer activity of phthalazine derivatives and assess their potential as selective inhibitors of PARP-1, which could lead to more effective cancer therapies with reduced toxicity. By leveraging green synthesis methods such as microwave-assisted reactions, researchers aim to streamline the development of these compounds while improving their pharmacological profiles.

While phthalazine derivatives hold considerable promise, several challenges remain in their synthesis to achieve higher efficiency, yield, and selectivity. Traditional methods for producing these compounds often involve lengthy reaction times, increased energy consumption, and limited control over reaction conditions, which can hinder their practical use. Moreover, the full therapeutic potential of phthalazine derivatives, particularly in their role as PARP-1 inhibitors for cancer treatment, has yet to be fully realized, and the underlying molecular mechanisms require further investigation.

There is a clear need for efficient and environmentally sustainable approaches to synthesizing phthalazine derivatives. Accordingly, one objective of the present disclosure is to explore the development and synthesis of phthalazine compounds using microwave-assisted methods that may circumvent the aforementioned stated drawbacks.

### SUMMARY

In an exemplary embodiment, a method of synthesizing a phthalazine compound by microwave irradiation is described. The method includes reacting phosphorus oxychloride, phosphorus pentachloride, and 4-(4-isopropyl-phenyl)-2H-phthalazin-1-one in an organic solvent to obtain a reaction mixture including 1-chloro-4-(4-isopropyl-phenyl)-phthalazine, and mixing at least one selected from the group including a hydrazine, an amine, an acetone, a dicarbonyl, a dinitrile, and a semicarbazide with the reaction mixture to obtain a final mixture, then microwave irradiating the final mixture at a power of 250 to 450 watt (W) for 5 to 30 minutes (min) to form the phthalazine compound.

In some embodiments, the phthalazine compound is at least one selected from the group consisting of

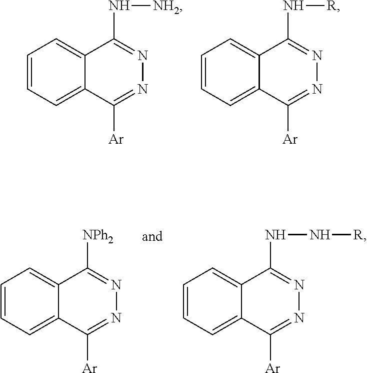

Ar is 4-isopropyl-phenyl, and R is a —CH₃, —C₆H₅, —C₁₀H₇, 4-(Me)-C₆H₄, 4-(OMe)-C₆H₄, 3,4-(Me)₂-C₆H₃, 3-(NO₂)—C₆H₄, 2,4-(NO₂)₂—C₆H₃, or —(CO)-2-thiophene.

In some embodiments, the phthalazine compound is at least one selected from the group consisting of

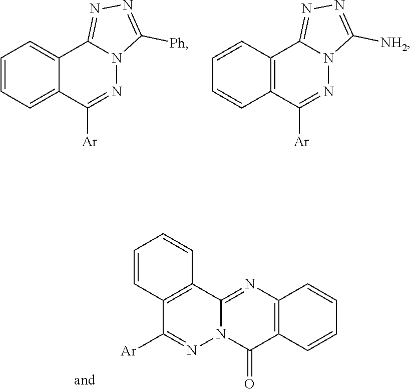

Ar is 4-isopropyl-phenyl.

In some embodiments, the reaction mixture is mixed with at least one semicarbazide selected from the group including semicarbazide hydrochloride, thiosemicarbazide, 4-phenylsemicarbazide, 4-methylsemicarbazide, 4-nitro semicarbazide, 1-acetylsemicarbazide, and 1-phenylsemicarbazide.

In some embodiments, the phthalazine compound is at least one selected from the group including

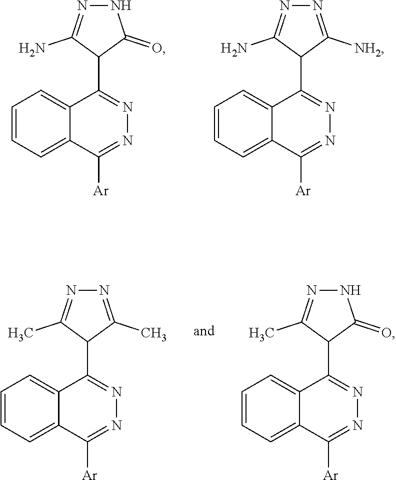

Ar is 4-isopropyl-phenyl.

In some embodiments, the reaction mixture is mixed with at least one dicarbonyls selected from the group consisting of acetylacetone, ethyl acetoacetate, dimethyl malonate.

In some embodiments, the organic solvent is benzene.

In some embodiments, the microwave irradiating includes microwave irradiating the final mixture at a power of 300 to 400 W for 9 to 20 min.

In some embodiments, the reaction mixture is mixed with at least one amine selected from the group including a methylamine, an aniline, a p-toluidine, a p-anisidine, a β-naphthylamine, an anthranilic acid, and a diphenylamine.

In some embodiments, the phthalazine compound is at least one selected from the group including 4-(4-isopropyl-phenyl)-phthalazin-1-yl]-hydrazine, N-[4-(4-isopropyl-phenyl)-phthalazin-1-yl]-N′-phenyl-hydrazine, N-[4-(4-isopropyl-phenyl)-phthalazin-1-yl]-N′-(3,4-dimethyl phenyl-hydrazine, N-[4-(4-isopropyl-phenyl)-phthalazin-1-yl]-N′-(3-nitro-phenyl)-hydrazine, N-[4-(4-isopropyl-phenyl)-phthalazin-1-yl]-N′-(2,4-dinitro-phenyl)-hydrazine, N-[4-(4-isopropyl-phenyl)-phthalazin-1-yl]-hydrazide-N′-thiophene-2-carboxylic acid, 6-(4-isopropyl-phenyl)-3-phenyl-[1,2,4]tria-zolo[3,4-a]-phthalazine, 6-(4-isopropyl-phenyl)-[1,2,4]triazolo[3,4-a]-phthalazin-3-yl-amine, 4-(4-isopropyl-phenyl)-phthalazin-1-yl]-methyl-amine, 4-(4-isopropyl-phenyl)-phthalazin-1-yl]-phenyl-amine, 4-(4-isopropyl-phenyl)-phthalazin-1-yl]-p-tolyl-amine, 4-(4-isopropyl-phenyl)-phthalazin-1-yl]-(4-methoxy-phenyl)-amine, 4-(4-isopropyl-phenyl)-phthalazin-1-yl]-naphthalen-2-yl-amine, 5-(4-isopropyl-phenyl)-6,6a,12-triaza-benzo[a]anthracen-7-one, 4-(4-isopropyl-phenyl)-phthalazin-1-yl]-diphenyl-amine, 1-(3,5-Dimethyl-4H-pyrazol-4-(4-isopropyl-phenyl)-phthalazine, 4-[4-(4-isopropyl-phenyl)-phthalazin-1-yl]-5-methyl-2,4-dihydro-pyrazol-3-one, 5-amino-4-[4-(4-isopropyl-phenyl)-phthala-zin-1-yl]-2,4-dihydro-pyrazol-3-one, and 4-[4-(4-isopropyl-phenyl)-phthalazin-1-yl]-4H-pyrazole-3,5-diamine.

In some embodiments, the microwave irradiating includes microwave irradiating the final mixture at a power of 300 to 400 W for 9 to 10 min.

In some embodiments, the hydrazine is at least one selected from the group including a hydrazine hydrate, a phenylhydrazine, a 3,4-dimethylphenylhydrazine, a 3-nitrophenylhydrazine, a 2,4-dinitrophenyl hydrazine, a 2-thiophene hydrazine, and a benzoyl hydrazine.

In another exemplary embodiment, a method of treating a cancer in a subject is described. The method includes contacting the cancer with a 0.01 to 50 micromolar (μM) solution of the phthalazine compound. The cancer is at least one cancer line selected from the group including Michigan Cancer Foundation-7 (MCF7), Human Colon Tumor 116 (HCT116), and Human Hepatocellular Carcinoma G2 (HepG2).

In some embodiments, the cancer is MCF7 and the phthalazine compound has a half-maximal inhibitory concentration (IC₅₀) of 5.5 μM or less.

In some embodiments, the cancer is HCT116 and the phthalazine compound has an IC₅₀ of 30 μM or less.

In some embodiments, the cancer is HepG2 and the phthalazine compound has an IC₅₀ of 6 μM or less.

In some embodiments, the cancer is MCF7 and the phthalazine compound has a selectivity index (SI) of 1 to 42.

In some embodiments, the cancer is HCT116 and the phthalazine compound has an SI of 1 to 68.

In some embodiments, the cancer is HepG2 and the phthalazine compound has an SI of 1 to 17.

In some embodiments, the phthalazine compound has a binding score of −10 to −5.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 shows a schematic flow chart of a method of synthesizing a phthalazine compound by microwave irradiation, according to certain embodiments.

FIG. 2 shows a schematic of compound design based on structure similarity with an anticancer drug, olaparib, according to certain embodiments.

FIG. 3 shows a synthesis scheme for the preparation of chlorophthalazine compound 2, according to certain embodiments.

FIG. 4 shows reaction schemes of chlorophthalazine compound 2 with various hydrazine derivatives, according to certain embodiments.

FIG. 5 shows reaction schemes of chlorophthalazine compound 2 with primary and secondary amines, according to certain embodiments.

FIG. 6 shows synthesis schemes for the preparation of phthalazine derivatives 18, 19, 20 and 21, according to certain embodiments.

FIG. 7 shows a dose response curve of compound 6 against Michigan Cancer Foundation-7 (MCF7), Human Colon Tumor 116 (HCT116), and Human Hepatocellular Carcinoma G2 (HepG2), and Medical Research Council cell strain 5 (MRC5) cells. [X-axis: dose, Y-axis: absorbance], according to certain embodiments.

FIG. 8 shows a schematic for comparative binding positions of compound 6, compound 13, compound 19, compound 20, and olaparib bound to poly (ADP-Ribose) polymerase 1 (PARP-1) (7aad) active site, according to certain embodiments.

FIG. 9A shows a schematic for surface representation of compound 6 bound to PARP-1 (7aad) active site, according to certain embodiments.

FIG. 9B shows a schematic for surface representation of compound 13 bound to PARP-1 (7aad) active site, according to certain embodiments.

FIG. 9C shows a schematic for surface representation of compound 19 bound to PARP-1 (7aad) active site, according to certain embodiments.

FIG. 9D shows a schematic for surface representation of compound 20 bound to PARP-1 (7aad) active site, according to certain embodiments.

FIG. 10A shows a schematic for binding interactions of compound 6 bound to PARP-1 (7aad) active site, according to certain embodiments.

FIG. 10B shows a schematic for binding interactions of compound 13 bound to PARP-1 (7aad) active site, according to certain embodiments.

FIG. 10C shows a schematic for binding interactions of compound 19 bound to PARP-1 (7aad) active site, according to certain embodiments.

FIG. 10D shows a schematic for binding interactions of compound 20 bound to PARP-1 (7aad) active site, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, like reference numerals designate identical or corresponding parts throughout the several views. Further, as used herein, the words ‘a,’ ‘an’ and the like generally carry a meaning of ‘one or more,’ unless stated otherwise.

Furthermore, the terms ‘approximately,’ ‘approximate,’ ‘about,’ and similar terms generally refer to ranges that include the identified value within a margin of 20%, 10%, or preferably 5%, and any values therebetween.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

As used herein, the term ‘compound’ refers to a chemical entity, regardless of its phase-solid, liquid, or gaseous-as well as its state-crude mixture, purified, or isolated.

As used herein, the term ‘functional group’ indicates specific groups of atoms within a molecular structure that are accountable for the characteristic chemical reactions and chemical properties of that structure. Suitable examples of functional groups include hydrocarbons, groups containing halogen, groups containing oxygen (alcohol, acid, ketone, aldehydes), groups containing nitrogen (nitrile, amines, amides), groups containing silicon (silanes) and groups containing phosphorus and sulfur all identifiable by a skilled person.

As used herein, the term ‘attach’ or ‘attachment’ refers to linking or uniting by a bond, link, force, or tie in order to keep two or more parts together, which encompasses either direct or indirect attachment such that, for example, a first compound is directly bound to a second compound or material, and the embodiments wherein one or more intermediate compounds, and in particular molecules, are disposed between the first compound and the second compound or material.

As used herein, the term ‘aromatic compounds’ or ‘aromatic rings’, as used herein, refers to hydrocarbon rings that, by the theory of Hückel, have a cyclic, delocalized (4n+2) pi-electron system. Non-limiting examples of aromatic compounds include benzene, benzene derivatives, compounds having at least one benzene ring in their chemical structure.

As used herein, the term ‘alkyl’, unless otherwise specified, refers to a straight, branched, or cyclic, saturated aliphatic fragment having 1 to 26 carbon atoms, (e.g., C1, C2, C3, C4, C5, C6, C7, C8, C9, C10, C11, etc.) and specifically includes, but is not limited to, methyl, ethyl, propyl, isopropyl, butyl, isobutyl, t-butyl, pentyl, isopentyl, neopentyl, hexyl, isohexyl, 3-methylpentyl, 2,2-dimethylbutyl, 2,3-dimethylbutyl, 2-ethylhexyl, heptyl, octyl, nonyl, 3,7-dimethyloctyl, decyl, undecyl, dodecyl, tridecyl, tetradecyl, pentadecyl, hexadecyl, heptadecyl, octadecyl, nonadecyl, eicosyl, guerbet-type alkyl groups (e.g., 2-methylpentyl, 2-ethylhexyl, 2-propylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl), as well as cyclic alkyl groups (cycloalkyls) such as cyclopropyl, cyclobutyl, cyclopentyl, cyclohexyl, norbornyl, and adamantyl.

As used herein, the term ‘amines’ refer to the compounds and functional groups that contain a basic nitrogen atom with a lone pair. Amines are formally derivatives of ammonia (NH₃), where one or more hydrogen atoms have been replaced by a substituent such as an alkyl or aryl group (these may respectively be called alkylamines and arylamines; amines in which both types of substituents are attached to one nitrogen atom may be called alkylarylamines). Amines may include amino acids, biogenic amines, trimethylamine, and aniline. Inorganic derivatives of ammonia are also called amines, such as monochloramine (NCIH₂).

As used herein, the term ‘aromatic amine’ refers to the organic compound including an aromatic ring attached to an amine. It is a broad class of compounds that encompasses anilines, but also many more complex aromatic rings and many amine substituents beyond NH₂.

As used herein, the term ‘subject’ refers to the organism, individual, or entity being studied or experimented on, e.g., a patient.

As used herein, the term ‘cancer’ refers to all types of cancer, neoplasm or malignant tumors found in mammals (e.g. humans), including leukemias, lymphomas, carcinomas, and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term ‘half-maximal inhibitory concentration (IC₅₀)’ refers to the concentration of a substance needed to reduce the activity of a specific biological target (such as a protein or enzyme) by 50%. It's a common metric in research to gauge how effective a compound is in inhibiting a particular biological function. A lower IC₅₀ signifies a stronger inhibitor, as it requires less of the substance to achieve the same level of inhibition.

As used herein, the term ‘selectivity index (SI)’ refers to the ratio that compares the effectiveness of a compound in inhibiting a specific target or biological activity to its toxicity toward non-target cells or tissues. A higher SI indicates better specificity and reduced harmful effects.

As used herein, the term ‘binding score’ refers to a numerical value a value that measures the affinity between a molecule, such as a ligand, and its target, like a protein or receptor. A higher binding score typically indicates a stronger and more stable interaction.

A weight percent of a component, unless specifically stated to the contrary, is based on the total weight of the formulation or composition in which the component is included. For example, if a particular element or component in a composition or article is said to have 5 weight percentage (wt. %), it is understood that this percentage is in relation to a total compositional percentage of 100%.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers. By way of general example, and without limitation, isotopes of hydrogen include deuterium and tritium, and isotopes of carbon include ¹³C and ¹⁴C. Isotopes of oxygen include ¹⁶O, ¹⁷O, and ¹⁸O. Isotopically-labeled compounds of the disclosure may generally be prepared by conventional techniques known to those skilled in the art or by processes analogous to those described herein, using an appropriate isotopically-labeled reagent in place of the non-labeled reagent otherwise employed.

Aspects of the present disclosure are directed towards microwave-assisted synthesis of a phthalazine compound for anticancer activity. This method offers the potential for reduced reaction times, improved yields, and enhanced product purity. The phthalazine derivatives show promising anticancer activity and selectively inhibit cancer cell growth and target poly (ADP-Ribose) polymerase 1 (PARP-1), offering potential for new therapies.

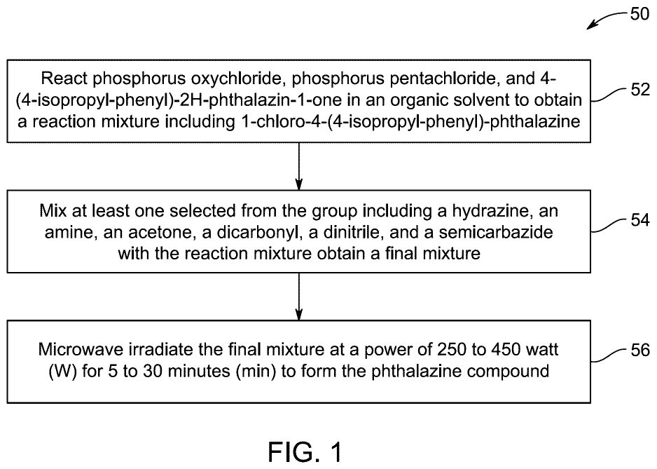

FIG. 1 illustrates a schematic flow chart of a method 50 of synthesizing a phthalazine compound by microwave irradiation. The order in which the method 50 is described is not intended to be construed as a limitation, and any number of the described method steps can be combined to implement the method 50. Additionally, individual steps may be removed or skipped from the method 50 without departing from the spirit and scope of the present disclosure.

At step 52, the method 50 includes reacting phosphorus oxychloride, phosphorus pentachloride, and 4-(4-isopropyl-phenyl)-2H-phthalazin-1-one in an organic solvent to obtain a reaction mixture including 1-chloro-4-(4-isopropyl-phenyl)-phthalazine. An organic solvent is a carbon-based substance employed for the dissolution of other substance(s). Exemplary organic solvents include, but are not limited to, ethanol, methanol, propanol, isopropanol (IPA), butanol, acetone, ethyl acetate, benzene, toluene, xylene, chloroform, dichloromethane (THF), tetrahydrofuran (THF), dimethyl sulfoxide (DMSO), dimethylformamide (DMF), acetonitrile, hexane, heptane, octane, nonane, decane, dioxane, diethyl ether, petroleum ether, naphtha, formamide, pyridine, nitrobenzene, anisole, and ethylene glycol. In a preferred embodiment, the organic solvent is benzene.

In some embodiments, phosphorus oxychloride and phosphorus pentachloride are present in a molar ratio of 1:5-5:1, preferably 1:4-4:1, preferably 1:3-3:1, preferably 1:2-2:1, and preferably 1:1. In a preferred embodiment, phosphorus oxychloride and phosphorus pentachloride are present in a molar ratio of 3:1. In some embodiments, phosphorus pentachloride and 4-(4-isopropyl-phenyl)-2H-phthalazin-1-one are present in a molar ratio of 1:5-5:1, preferably 1:4-4:1, preferably 1:3-3:1, preferably 1:2-2:1, and preferably 1:1. In a preferred embodiment, phosphorus pentachloride and 4-(4-isopropyl-phenyl)-2H-phthalazin-1-one are present in a molar ratio of 1:1. In some embodiments, phosphorus oxychloride and 4-(4-isopropyl-phenyl)-2H-phthalazin-1-one are present in a molar ratio of 1:5-5:1, preferably 1:4-4:1, preferably 1:3-3:1, preferably 1:2-2:1, and preferably 1:1. In a preferred embodiment, phosphorus oxychloride and 4-(4-isopropyl-phenyl)-2H-phthalazin-1-one are present in a molar ratio of 3:1.

In some embodiments, the reaction may be done via stirring, swirling, sonicating, or refluxing. In a preferred embodiment, phosphorus oxychloride, phosphorus pentachloride, and 4-(4-isopropyl-phenyl)-2H-phthalazin-1-one are reacting via refluxing to obtain a reaction mixture including 1-chloro-4-(4-isopropyl-phenyl)-phthalazine. Refluxing is a process that involves vapor condensation and the return of the condensate to the source system. Refluxing facilitates the continuous reaction by preventing the loss of volatile solvents or reagents, thereby ensuring that the reaction components remain in the reaction flask for the duration of the process.

At step 54, the method 50 includes mixing at least one selected from the group including a hydrazine, an amine, an acetone, a dinitrile, and a semicarbazide with the reaction mixture obtain a final mixture. In some embodiments, the reaction mixture is mixed with at least one semicarbazide selected from the group including semicarbazide hydrochloride, thiosemicarbazide, 4-phenylsemicarbazide, 4-methylsemicarbazide, 4-nitro semicarbazide, 1-acetylsemicarbazide, and 1-phenylsemicarbazide. In a preferred embodiment, the reaction mixture is mixed with semicarbazide hydrochloride.

In some embodiments, the reaction mixture is mixed with at least one amine selected from the group including a methylamine, an aniline, a p-toluidine, a p-anisidine, a f-naphthylamine, an anthranilic acid, and a diphenylamine. In some embodiments, the hydrazine is at least one selected from the group including a hydrazine hydrate, a phenylhydrazine, a 3,4-dimethylphenylhydrazine, a 3-nitrophenylhydrazine, a 2,4-dinitrophenyl hydrazine, a 2-thiophene hydrazine, and a benzoyl hydrazine.

At step 56, the method 50 includes microwave irradiating the final mixture at a power of 250-450 watt (W), preferably 260-440 W, preferably 270-430 W, preferably 280-420 W, preferably 290-410 W, preferably 300-400 W, preferably 310-390 W, preferably 320-380 W, preferably 330-370 W, preferably 340-360 W for 5-30 minutes (min), 5-30 min, preferably 6-29 min, preferably 7-28 min, preferably 8-27 min, preferably 9-26 min, preferably 10-25 min, preferably 11-24 min, preferably 12-23 min, preferably 13-22 min, preferably 14-21 min, preferably 15-20 min, preferably 16-19 min, and preferably 17-18 min to form the phthalazine compound.

In some embodiments, the microwave irradiating includes microwave irradiating the final mixture at a power of 300-400 W, preferably 310-390 W, preferably 320-380 W, preferably 330-370 W, and preferably 340-360 W for 9-20 min, preferably 10-19 min, preferably 11-18 min, preferably 12-17 min, preferably 13-16 min, and preferably 14-15 min. In some embodiments, the microwave irradiating includes microwave irradiating the final mixture at a power of 00-400 W, preferably 310-390 W, preferably 320-380 W, preferably 330-370 W, and preferably 340-360 W for 9-10 min, and preferably 9.5 min.

Microwave irradiating includes exposing the mixture to microwave radiation, which heats the components rapidly. This process facilitates the formation of the phthalazine compound by promoting chemical reactions through efficient energy transfer. Microwave-assisted synthesis has emerged as an advantageous method for preparing heterocyclic compounds, including phthalazine derivatives. This technique provides several benefits such as accelerated reaction rates, improved yields, reduced reaction times, and milder conditions, contributing to more efficient and sustainable synthesis. The greener approach of microwave-assisted methods, which requires lower energy consumption, has also been widely recognized for its environmental advantages. Previous studies have demonstrated successful syntheses of phthalazine derivatives through microwave irradiation, achieving high yields and enhanced purity.

In some embodiments, the phthalazine compound is at least one selected from the group consisting of

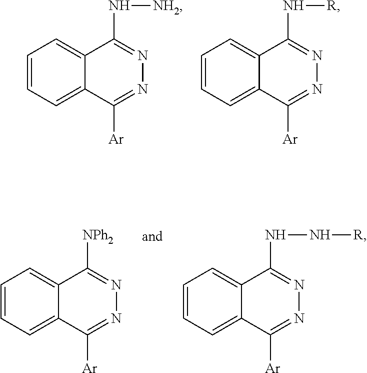

Ar may be 4-isopropyl-phenyl, and R is a —CH₃, —C₆H₅, —C₁₀H₇, 4-(Me)-C₆H₄, 4-(OMe)-C₆H₄, 3,4-(Me)₂-C₆H₃, 3-(NO₂)—C₆H₄, 2,4-(NO₂)₂—C₆H₃, or —(CO)-2-thiophene.

In some embodiments, the phthalazine compound is at least one selected from the group consisting of

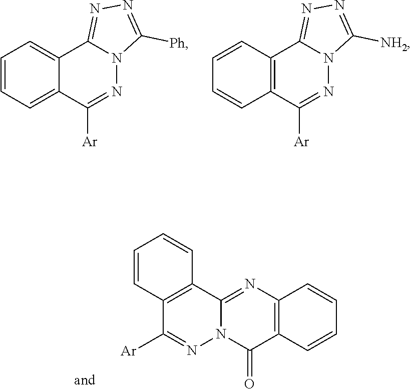

wherein Ar is 4-isopropyl-phenyl.

In some embodiments, the phthalazine compound is at least one selected from the group consisting of

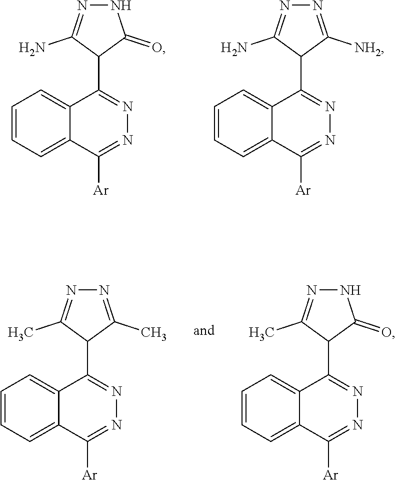

wherein Ar is 4-isopropyl-phenyl.

In some embodiments, the phthalazine compound is at least one selected from the group including 4-(4-isopropyl-phenyl)-phthalazin-1-yl]-hydrazine, N-[4-(4-isopropyl-phenyl)-phthalazin-1-yl]-N′-phenyl-hydrazine, N-[4-(4-isopropyl-phenyl)-phthalazin-1-yl]-N′-(3,4-dimethyl phenyl-hydrazine, N-[4-(4-isopropyl-phenyl)-phthalazin-1-yl]-N′-(3-nitro-phenyl)-hydrazine, N-[4-(4-isopropyl-phenyl)-phthalazin-1-yl]-N′-(2,4-dinitro-phenyl)-hydrazine, N-[4-(4-isopropyl-phenyl)-phthalazin-1-yl]-hydrazide-N′-thiophene-2-carboxylic acid, 6-(4-isopropyl-phenyl)-3-phenyl-[1,2,4]tria-zolo[3,4-a]-phthalazine, 6-(4-isopropyl-phenyl)-[1,2,4]triazolo[3,4-a]-phthalazin-3-yl-amine, 4-(4-isopropyl-phenyl)-phthalazin-1-yl]-methyl-amine, 4-(4-isopropyl-phenyl)-phthalazin-1-yl]-phenyl-amine, 4-(4-isopropyl-phenyl)-phthalazin-1-yl]-p-tolyl-amine, 4-(4-isopropyl-phenyl)-phthalazin-1-yl]-(4-methoxy-phenyl)-amine, 4-(4-isopropyl-phenyl)-phthalazin-1-yl]-naphthalen-2-yl-amine, 5-(4-isopropyl-phenyl)-6,6a,12-triaza-benzo[a]anthracen-7-one, 4-(4-isopropyl-phenyl)-phthalazin-1-yl]-diphenyl-amine, 1-(3,5-Dimethyl-4H-pyrazol-4-(4-isopropyl-phenyl)-phthalazine, 4-[4-(4-isopropyl-phenyl)-phthalazin-1-yl]-5-methyl-2,4-dihydro-pyrazol-3-one, 5-amino-4-[4-(4-isopropyl-phenyl)-phthala-zin-1-yl]-2,4-dihydro-pyrazol-3-one, and 4-[4-(4-isopropyl-phenyl)-phthalazin-1-yl]-4H-pyrazole-3,5-diamine.

In some embodiments, a method of treating a cancer in a subject is described. The method includes contacting the cancer with a 0.01-50 micromolar (μM), preferably 0.05-49 μM, preferably 0.1-48 μM, preferably 0.5-47 μM, preferably 1-46 μM, preferably 2-45 μM, preferably 3-44 μM, preferably 4-43 μM, preferably 5-42 μM, preferably 6-41 μM, preferably 7-40 μM, preferably 8-39 μM, preferably 9-38 μM, preferably 10-37 μM, preferably 11-36 μM, preferably 12-35 μM, preferably 13-34 μM, preferably 14-33 μM, preferably 15-32 μM, preferably 16-31 μM, preferably 17-30 μM, preferably 18-29 μM, preferably 19-28 μM, preferably 20-27 μM, preferably 21-26 μM, preferably 22-25 μM, and preferably 23-24 μM solution of the phthalazine compound. The cancer is at least one cancer line selected from the group including Michigan Cancer Foundation-7 (MCF7), Human Colon Tumor 116 (HCT116), and Human Hepatocellular Carcinoma G2 (HepG2).

In some embodiments, the cancer is MCF7 and the phthalazine compound has a IC₅₀ of 5.5 μM or less, preferably 5 μM, preferably 4.5 μM, preferably 4 μM, preferably 3.5 μM, preferably 3 μM, preferably 2.5 μM, preferably 2 μM, preferably 1.5 μM, preferably 1 μM, preferably 0.9 μM, preferably 0.8 μM, preferably 0.7 μM, preferably 0.6 μM, preferably 0.5 μM, preferably 0.4 μM, preferably 0.3 μM, preferably 0.2 μM, preferably 0.1 μM, preferably 0.09 μM, preferably 0.08 μM, preferably 0.07 μM, preferably 0.06 μM, preferably 0.05 μM, and preferably 0.04 μM.

In some embodiments, the cancer is HCT116 and the phthalazine compound has an IC₅₀ of 30 μM or less, preferably 29 μM, preferably 28 μM, preferably 27 μM, preferably 26 μM, preferably 25 μM, preferably 24 μM, preferably 23 μM, preferably 22 μM, preferably 21 μM, preferably 20 μM, preferably 19 μM, preferably 18 μM, preferably 17 μM, preferably 16 μM, preferably 15 μM, preferably 14 μM, preferably 13 μM, preferably 12 μM, preferably 11 μM, preferably 10 μM, preferably 9 μM, preferably 8 μM, preferably 7 μM, preferably 6 μM, preferably 5 μM, preferably 4 μM, preferably 3 μM, preferably 2 μM, preferably 1 μM, and preferably 0.1 μM.

In some embodiments, the cancer is HepG2 and the phthalazine compound has an IC₅₀ of 5 μM or less, preferably 4 μM, preferably 3 μM, preferably 2 μM, preferably 1 μM, preferably 0.5 μM and preferably 0.1 μM.

In some embodiments, the cancer is MCF7 and the phthalazine compound has a SI of 1-42, preferably 2-41, preferably 3-40, preferably 4-39, preferably 5-38, preferably 6-37, preferably 7-36, preferably 8-35, preferably 9-34, preferably 10-33, preferably 11-32, preferably 12-31, preferably 13-30, preferably 14-29, preferably 15-28, preferably 16-27, preferably 17-26, preferably 18-25, preferably 19-24, preferably 20-23, and preferably 21-22.

In some embodiments, the cancer is HCT116 and the phthalazine compound has an SI of 1-68, preferably 2-67, preferably 3-66, preferably 4-65, preferably 5-64, preferably 6-63, preferably 7-62, preferably 8-61, preferably 9-60, preferably 10-59, preferably 11-58, preferably 12-57, preferably 13-56, preferably 14-55, preferably 15-54, preferably 16-53, preferably 17-52, preferably 18-51, preferably 19-50, preferably 20-49, preferably 21-48, preferably 22-47, preferably 23-46, preferably 24-45, preferably 25-44, preferably 26-43, preferably 27-42, preferably 28-41, preferably 29-40, preferably 30-39, preferably 31-38, preferably 32-37, preferably 33-36, and preferably 34-35.

In some embodiments, the cancer is HepG2 and the phthalazine compound has an SI of 1-17, preferably 2-16, preferably 3-15, preferably 4-14, preferably 5-13, preferably 6-12, preferably 7-11, and preferably 8-10.

In some embodiments, the phthalazine compound has a binding score of −10 to −5, preferably −9 to −6, and preferably −8 to −7.

### EXAMPLES

The following examples demonstrate a method of synthesizing a phthalazine compound by microwave irradiation for anticancer activity. The examples are provided solely for illustration and are not to be construed as limitations of the present disclosure, as many variations thereof are possible without departing from the spirit and scope of the present disclosure.

### Example 1: Materials and Methods

All the solvents and reagents used were purchased from Sigma-Aldrich Chemicals, USA. The microwave-assisted method is carried out by using a CEM microwave (MARS 6 Synthesis) parallel and Scale-up microwave synthesizer by CEM company in the USA, at 300-400 W and 70-78.37 degrees Celsius (° C.). Melting points (mp) determined via the Stuart SMP30 Digital Advanced MP apparatus are uncorrected. Fourier transform infrared spectroscopy (FT-IR) spectra were recorded on an infrared (IR) Affinity spectrophotometer from Shimadzu. The sample prepared on glass plate contains solid KBr. Nuclear magnetic resonance (NMR) spectra were recorded by a BRUKER spectrometer (400 MHZ) by using dimethylsulphoxide (DMSO-d) as solvent, chemical shifts (δ) in part per million (ppm) using tetramethylsilane (TMS) as internal standard. The mass spectra were carried out on liquid chromatography-mass spectrometry (LC/MS) spectroscopy (LCMS-8040 Shimadzu, model CAT-30A). Elemental microanalysis was done on the Carlo Erba analyzer model 110.

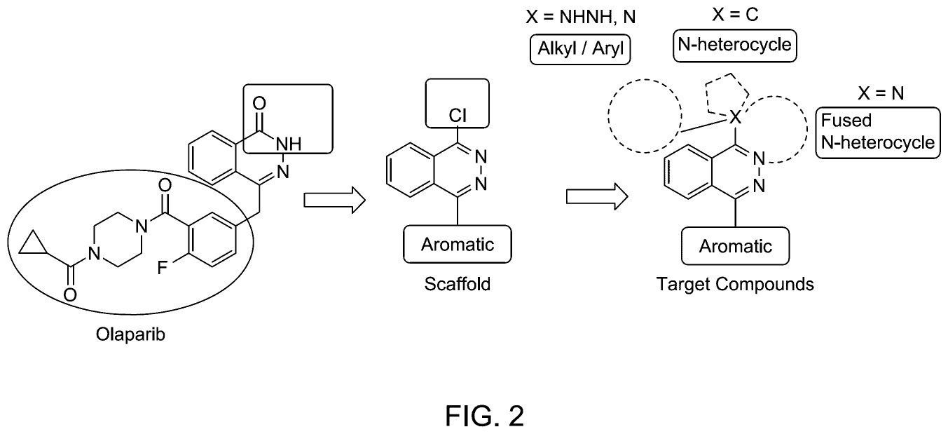

The synthesized compounds may have moieties in common with Olaparib, a poly(ADP-ribose) polymerase (PARP) inhibitor. The compounds of the present disclosure have enhanced target selectivity and reduced toxicity by incorporating a simultaneous 1,4-substitution pattern along with an amino group at the 4-position. This strategic modification leads to better pharmacological properties, offering a promising alternative to existing therapies with reduced adverse effects (FIG. 2). PARP is an enzyme involved in DNA repair. PARP inhibitors have shown promising results in both clinical trials and practice for the treatment of ovarian, breast, prostate, and pancreatic cancers.

### Example 2: Synthesis of 1-chloro-4-(4-isopropyl-phenyl)-phthalazine (2)

Compound 1 [4-(4-isopropyl-phenyl)-2H-phthalazin-1-one]was confirmed before, as in a previous study (See: Abubshait, S. A., Abubshait, H. A., Almalih, R., Gomaa, M. S., Nawaz. M., Ababutain, I. M., & Alghamdi, A. 1. (2022). Microwave-assisted synthesis of phthalazinone derivatives with biological activity and in silico antiproliferative studies. ChemistrySelect, 7(46), e202203925, incorporated herein by reference in its entirety). A mixture of 1 (2.64 grams (g), 10 millimoles (mmol)) dissolved in (30 milliliters (mL)) of dry benzene, phosphorus oxychloride (3.00 mL, 30 mmol), and phosphorus pentachloride (2.08 g, 10 mmol), under the air of nitrogen gas, was refluxed (30 minutes (min)). Then the mixture poured in crushed ice with sodium bicarbonate (NaHCO₃) to give compound 2 after filtered and crystallized from dry benzene to give beige powder; mp.: 240-242° C.; FT-IR (KBr, ν, cm⁻¹): 2922, 2865 (—CH, —CH₃), 3068-3010 (═CH, Ar), 1635, 1448 (C═C); ¹H NMR, δ (ppm) in DMSO-d₆: 1.28 (d, 6H, 2CH₃), 2.98 (m, 1H, —CH(CH₃)₂), 7.24-8.54 (m, 8H, —CH—, ArH); ¹³C NMR, δ (ppm) in DMSO-d₆: 23.91 (2C, —CH₃), 30.9 (1C, —CH(CH₃)₂), 126.7, 127.5, 128.5, 132.0, 148.0 (10C, ═CH—, Ar), 130.6, 134.0, 151.6 (3C, ═CH—, pyridazine), 171 (1C, —C—Cl); MS (m/z): 282.09 (M⁺,100.0), 283.10 (M⁺+1, 19.1); Analytical calculation (Anal. calcd.) for C₁₇H₁₅ClN₂/(282.77): C, 72.21; H, 5.35; N, 9.91; Cl, 12.54%. found: C, 72.22; H, 5.36; N, 9.92; Cl, 12.53%.

### Example 3: Synthesis of Hydrazine Derivatives (Compounds 3-10)

A mixture of 2 (2.84 g, 10 mmol) and hydrazine hydrate (0.65 mL, 20 mmol), and/or (10 mmol) phenyl hydrazine (0.98 mL), 3,4-dimethylphenylhydrazine hydrochloride (1.72 g), 3-nitrophenylhydrazine hydrochloride (1.89 g), 2,4-dinitrophenyl hydrazine (1.98 g), and 2-thiophene carboxylic hydrazide, benzoyl hydrazine (1.36 g), and semicarbazide hydrochloride (1.12 g) in (20 mL) dry benzene with triethylamine (Et₃N) (1 mL) was refluxed at 60° C. for 5-6 hours (h) (microwave (MW), 9-25 min). After that the precipitated products were collected, washed with cold benzene, dried, and then recrystallized from benzene.

### Example 4: [4-(4-isopropyl-phenyl)-phthalazin-1-yl]-hydrazine (3)

White powder; mp.: 260-263° C.; FT-IR (KBr, ν, cm⁻¹): 2956, 2868 (—CH, —CH₃), 3064-3010 (═CH, Ar), 1606, 1506 (C═C), 3304-3273 (—NH—NH₂); ¹H NMR, δ (ppm) in DMSO-d₆: 1.24 (d, 6H, 2CH₃), 3.47 (m, 1H, —CH(CH₃)₂), 7.34-8.75 (m, 8H, —CH—, Ar-H), 12.03, 13.01 (br, 3H, —NH—NH₂); ¹³C NMR, δ (ppm) in DMSO-d₆: 24.27 (2C, —CH₃), 33.7 (1C, —CH(CH₃)₂), 125.6, 127.5, 128.7, 132.0, 143.0 (10C, ═CH—, Ar), 130.8, 135.0, 155.6 (3C, ═C—, pyridazine), 168.0 (1C, ═C—NH—NH₂); MS (m/z): 278.15 (M⁺, 100.0), 279.16 (M⁺+1, 56.0); Anal. calcd. for C₁₇H₁₈N₄/(278.35); C, 73.35; H, 6.52; N, 20.13%. found: C, 73.33; H, 6.53; N, 20.14%.

### Example 5: N-[4-(4-isopropyl-phenyl)-phthalazin-1-yl]-N′-phenyl-hydrazine (4)

Brown powder; mp.:147-149° C.; FT-IR (KBr, ν, cm⁻¹): 2957, 2862 (—CH, —CH₃), 3050-3019 (═CH, Ar), 1606, 1491 (C═C), 3320-3210 (—NH—NH); ¹H NMR, δ (ppm) in DMSO-d₆: 2.48 (d, 6H, 2CH₃), 3.42 (m, 1H, —CH(CH₃)₂), 6.71-8.55 (m, 13H, —CH—, ArH), 11.86 (br, 2H, —NH—NH); ¹³C NMR, δ (ppm) in DMSO-d₆: 24.4 (2C, —CH₃), 33.7 (1C, —CH(CH₃)₂), 126.4, 128.4, 128.7, 132.0 (15C, ═CH—, Ar), 130.0, 134.0, 155.6 (3C, ═C—, pyridazine), 147.3, 166.0 (2C, ═C—NH—NH—C═); MS (m/z): 354.18 (M⁺,100.0), 355.19 (M⁺+1, 25.1); Anal. calcd. for C₂₃H₂₂N₄/(354.45): C, 77.94; H, 6.26; N, 15.81%. found: C, 78.00; H, 6.27; N, 15.83%.

### Example 6: N-[4-(4-isopropyl-phenyl)-phthalazin-1-yl]-N′-(3,4-dimethyl phenyl-hydrazine (5)

Light brown powder; mp.:115-116° C.; FT-IR (KBr, ν, cm⁻¹): 2956, 2868 (—CH, —CH₃), 3060-3022 (═CH, Ar), 1606, 1475 (C═C), 3320-3208 (—NH—NH); ¹H NMR, δ (ppm) in DMSO-d₆: 2.09 (d, 6H, 2CH₃), 2.50 (s, 6H, 2CH₃), 3.43 (m, 1H, —CH(CH₃)₂), 7.2-8.00 (m, 11H, —CH—, ArH), 8.40 (br, 2H, —NH—NH—); ¹³C NMR, δ (ppm) in DMSO-d₆: 8.9 (2C, —CH₃), 20.0 (2C, —CH₃), 39.5 (1C, —CH(CH₃)₂), 123.9, 127.8, 128.7, 130.4 (15C, ═CH—, Ar), 130.0, 135.0, 155.6 (3C, ═CH—, pyridazine), 167.1, 145.2 (2C, ═C—NH—NH—C═); MS (m/z): 382.22 (M⁺,100.0), 381.22 (M⁺+1, 47.7); Anal. calcd. for C₂₅H₂₆N₄/(382.50); C, 78.50; H, 6.85; N, 14.65%. found: C, 78.48; H, 6.85; N, 14.67%.

### Example 7: N-[4-(4-isopropyl-phenyl)-phthalazin-1-yl]-N′-(3-nitro-phenyl)-hydrazine (6)

Light orange powder; mp.: 196-198° C.; FT-IR (KBr, ν, cm⁻¹): 2933, 2850 (—CH, —CH₃), 3088-3010 (═CH, Ar), 1599, 1457 (C═C), 1550,1372 (—NO₂), 3321-3319 (—NH—NH); ¹H NMR, δ (ppm) in DMSO-d₆: 1.26 (d, 6H, 2CH₃), 3.42 (m, 1H, —CH(CH₃)₂), 7.35-8.91 (m, 12H, —CH—, ArH), 10.33, 12.80 (br, 2H, —NH—NH—); ¹³C NMR, δ (ppm) in DMSO-d₆: 24.3 (2C, —CH₃), 32.7 (1C, —CH(CH₃)₂), 108.4, 116, 121, 128.8, 132.0 (14C, ═CH—, Ar), 130.0, 133.0, 155.6 (3C, ═CH—, pyridazine), 148.9 (1C, ═CH—NO₂), 147.5, 169.0 (2C, ═C—NH—NH—C═); MS (m/z): 399.17 (M⁺, 62.4), 401.17 (M⁺+2, 16.1), 401.18 (M⁺2, 3.2); Anal. calcd. for C₂₃H₂₁N₅O₂/(399.45): C, 69.16; H, 5.30; N, 17.53%. found: C, 69.16; H, 5.28; N, 17.52%.

### Example 8: N-[4-(4-isopropyl-phenyl)-phthalazin-1-yl]-N′-(2,4-dinitro-phenyl)-hydrazine (7)

Brown powder; mp.: 186-188° C.; FT-IR (KBr, ν, cm⁻¹): 2930, 2853 (—CH, —CH₃), 3078-3010 (═CH, Ar), 1597, 1458 (C═C), 1550, 1370 (—NO₂), 3421-3319 (—NH—NH); ¹H NMR, δ (ppm) in DMSO-d₆: 1.29 (d, 6H, 2CH₃), 3.12 (m, 1H, —CH(CH₃)₂), 7.19-8.90 (m, 11H, —CH—, ArH), 10.33, 12.81 (br, 2H, —NH—NH—); ¹³C NMR, δ (ppm) in DMSO-d₆: 24.4 (2C, —CH₃), 31.7 (1C, —CH(CH₃)₂), 113.4, 117, 126.7, 127, 133.7, 148.7 (15C, ═CH—, Ar), 143.6 (1C, ═CH—, pyridazine), 139.9 (2C, ═CH—NO₂), 143.5, 166.0 (2C, ═C—NH—NH—C═); MS (m/z): 444.15 (M⁺, 62.4), 446.17 (M++2, 16.1), Anal. calcd. for C₂₃H₂₀N₆O₄/(444.44): C, 62.16; H, 4.54; N, 18.91%. found: C, 62.19; H, 5.58; N, 18.82%.

### Example 9: N-[4-(4-isopropyl-phenyl)-phthalazin-1-yl]-hydrazide-N′-thiophene-2-carboxylic Acid (8)

Light brown powder; mp.: 190-192° C.; FT-IR (KBr, ν, cm⁻¹): 2930, 2852 (—CH, —CH₃), 3079-3010 (═CH, Ar), 1599, 1457 (C═C), 3421-3319 (—NH—NH); 1680 (C═O); ¹H NMR, δ (ppm) in DMSO-d₆: 1.26 (d, 6H, 2CH₃), 3.42 (m, 1H, —CH(CH₃)₂), 7.06-7.93 (m, 11H, —CH—, ArH), 10.33, 12.80 (br, 2H, —NH—NH—); ¹³C NMR, δ (ppm) in DMSO-d₆: 24.4 (2C, —CH₃), 31.7 (1C, —CH(CH₃)₂), 119, 126, 128.5, 133.0, 136.7 (13C, ═CH—, Ar), 117.0, 127.0, 143.3 (3C, ═CH—, pyridazine), 145.5 (1C, —C—CO) 167.3 (1C, —C5O), 166.0 (1C, NH—NH—C═); MS (m/z): 388.49 (M⁺, 100), 389.14.17 (M⁺+1, 15.1), Anal. calcd. For C₂₂H₂₀N₄OS/(388.49): C, 68.02; H, 5.19; N, 14.42; S, 8.25%. found: C, 68.05; H, 5.20; N, 14.52, S, 8.22%.

### Example 10: 6-(4-isopropyl-phenyl)-3-phenyl-[1,2,4]triazolo[3,4-a]-phthalazine (9)

Light orange powder; mp.: 186-188° C.; FT-IR (KBr, ν, cm⁻¹): 2928, 2850 (—CH, —CH₃), 3086-3011 (═CH, Ar), 1597, 1459 (C═C); ¹H NMR, δ (ppm) in DMSO-d₆: 1.29 (d, 6H, 2CH₃), 3.12 (m, 1H, —CH(CH₃)₂), 7.19-7.93 (m, 13H, —CH—, ArH); ¹³C NMR, δ (ppm) in DMSO-d₆: 24.2 (2C, —CH₃), 31.7 (1C, —CH(CH₃)₂), 126.1, 128.8, 129.0, 133.0, 136.5 (18C, ═CH—, Ar, pyridazine), 127.2 (2C, ═CH—, pyridazine), 148.1 (1C, ═CH—, triazole), 152.1 (2C, ═C—N—N—C═); MS (m/z): 364.44 (M⁺, 100), 365.40 (M⁺+1, 18.1), Anal. calcd. for C₂₄H₂₀N₄/(364.44): C, 79.10; H, 5.53; N, 15.37%. found: C, 79.14; H, 5.55; N, 15.32%.

### Example 11: 6-(4-isopropyl-phenyl)-[1,2,4]triazolo[3,4-a]-phthalazin-3-yl-amine (10)

Light yellow; mp.: 176-178° C.; FT-IR (KBr, ν, cm⁻¹): 2932, 2853 (—CH, —CH₃), 3090-3030 (═CH, Ar), 1595, 1457 (C═C), 3386-3330 (—NH₂); ¹H NMR, δ (ppm) in DMSO-d₆: 1.29 (d, 6H, 2CH₃), 3.12 (m, 1H, —CH(CH₃)₂), 7.19-7.93 (m, 8H, —CH—, ArH), 10.3 (br, 2H, —NH₂); ¹³C NMR, δ (ppm) in DMSO-d₆: 24.3 (2C, —CH₃), 31.7 (1C, —CH(CH₃)₂), 126.7, 133.0, 148.7 (10C, ═CH—, Ar), 127.0, 125.1 (4C, ═CH—, pyridazine), 148.9 (1C, ═C—NH₂); MS (m/z): 303.36 (M⁺, 65.4), 305.16 (M⁺+2, 16.1), Anal. calcd. for C₁₈H₁₇N₅/(303.36): C, 71.27; H, 5.65; N, 23.09%. found: C, 71.29; H, 5.69; N, 23.10%.

### Example 12: Synthesis of Compound (11-17)

A mixture of (2.84 g, 10 mmol) of 2 and (10 mmol) of methyl-amine (0.31 mL), aniline (0.93 mL), p-toluidine (1.07 g), p-anisidine (1.23 g), P-naphthyl amine (1.43 g), anthranilic acid (1.37 g) and diphenylamine (1.69 g) in dry benzene (30 mL) placed in an oil bath and refluxed 4-6 h at 80° C. and (MW, 9-20 min). The residues were filtered to crystal line from, the mixture was extracted with ether, and ether layer was dried with anhydrous MgSO₄. Finally, the residue was distilled off to be crystallized from a suitable solvent to give 11-17.

### Example 13: [4-(4-isopropyl-phenyl)-phthalazin-1-yl]-methyl-amine (11)

Beige powder; mp.:160-162° C.; FT-IR (KBr, ν, cm⁻¹): 2970, 2868 (—CH, —CH₃), 3070-3010 (═CH, Ar), 1607, 1456 (C═C), 3450 (—NH), 1188 (—C—N); ¹H NMR, δ (ppm) in DMSO-d₆: 1.27 (d, 6H, 2CH₃), 2.31 (d, 3H, —CH₃), 3.16 (m, 1H, —CH(CH₃)₂), 7.19-8.03 (m, 8H, —CH—, ArH), 10.57 (br, 1H, —NH—); ¹³C NMR, δ (ppm) in DMSO-d₆: 24.1 (2C, —CH₃), 35.3 (1C, —CH₃), 31.5 (1C, —CH(CH₃)₂), 126.7, 127.4, 132.1, 133.4 (10C, ═CH—, Ar), 117, 127.1, 143.1 (3C, ═CH—, pyridazine), 166.0 (1C, ═C—NH—); MS (m/z): 277.36 (M⁺, 98.0); Anal. calcd. for C₁₈H₁₉N₃/(277.36): C, 77.95; H, 6.90; N, 15.15%. found: C, 77.93; H, 6.87; N, 15.17%.

### Example 14: [4-(4-isopropyl-phenyl)-phthalazin-1-yl]-phenyl-amine (12)

Beige powder; mp.:150-151° C.; FT-IR (KBr, ν, cm⁻¹): 2955, 2870 (—CH, —CH₃), 3061-3010 (═CH, Ar), 1607, 1456 (C═C), 3450 (—NH), 1188 (—C—N); ¹H NMR, δ (ppm) in DMSO-d₆: 1.27 (d, 6H, 2CH₃), 3.46 (m, 1H, —CH(CH₃)₂), 6.58-8.33 (m, 13H, —CH—, ArH), 11.57 (br, 1H, —NH—); ¹³C NMR, δ (ppm) in DMSO-d₆: 24.3 (2C, —CH₃), 31.7 (1C, —CH(CH₃)₂), 115.4, 117.0, 126.9, 128.4, 129.4, 133.1, 147.1 (15C, ═CH—, Ar), 131.0, 133.3, 151.1 (3C, ═CH—, pyridazine), 166.2, 166.0 (2C, ═C—NH—C═); MS (m/z): 339.17 (M⁺, 61.0); Anal. calcd. for C₂₃H₂₁N₃/(339.43): C, 81.38; H, 6.24; N, 12.38%. found: C, 81.40; H, 6.22; N, 12.40%.

### Example 15: [4-(4-isopropyl-phenyl)-phthalazin-1-yl]-p-tolyl-amine (13)

Beige powder; mp.:140-141° C.; FT-IR (KBr, ν, cm⁻¹): 2965, 2868 (—CH, —CH₃), 3050-3020 (═CH, Ar), 1604, 1479 (C═C), 3450 (—NH), 1170 (—C—N); ¹H NMR, δ (ppm) in DMSO-d₆: 2.18 (d, 6H, 2CH₃), 2.51 (s, 3H, 2CH₃), 3.54 (m, 1H, —CH(CH₃)₂), 6.71-8.07 (m, 12H, —CH—, ArH), 9.0 (br, 1H, —NH—); ¹³C NMR, δ (ppm) in DMSO-d₆: 20.7 (1C, —CH₃), 24.3 (2C, —CH₃), 34.7 (1C, —CH(CH₃)₂), 117.0, 128.4, 129.4, 133.1, 141.1 (15C, ═CH—, Ar), 130.0, 133.3, 151.1 (3C, ═CH—, pyridazine), 168.2, 169.0 (2C, ═C—NH—C═); MS (m/z): 353.19 (M⁺, 100.0), 350.19 (M⁺−3, 7.8); Anal. calcd. for C₂₄H₂₃N₃/(353.46): C, 81.55; H, 6.56; N, 11.89%. found: C, 81.54; H, 6.56; N, 11.90%.

### Example 16: [4-(4-isopropyl-phenyl)-phthalazin-1-yl]-(4-methoxy-phenyl)-amine (14)

Light purple powder; mp.: 126-128° C.; FT-IR (KBr, ν, cm⁻¹): 2960, 2868 (—CH, —CH₃), 3060-3020 (═CH, Ar), 1600, 1475 (C═C), 1270 (—C—O—C), 3450 (—NH), 1170 (—C—N); ¹H NMR, δ (ppm) in DMSO-d₆: 1.27 (d, 6H, 2CH₃), 3.46 (m, 1H, —CH(CH₃)₂), 1.27 (s, 3H, —OCH₃), 6.77-8.07 (m, 12H, —CH—, ArH), 11.34 (br, 1H, —NH—); ¹³C NMR, δ (ppm) in DMSO-d₆: 24.3 (2C, —CH₃), 31.7 (1C, —CH(CH₃)₂), 55.8 (1C, —OCH₃), 115.4, 118, 125.6, 128.4, 129.4, 133.1, 147.1 (14C, ═CH—, Ar), 131.0, 133.3, 151.1 (3C, ═CH—, pyridazine), 154.0 (1C, ═C—OCH₃), 139.0, 169.0 (2C, ═C—NH—C═); MS (m/z): 369.18 (M⁺, 100.0), 370.19 (M⁺+1, 27.1); Anal. calcd. for C₂₄H₂₃N₃O/(369.46): C, 78.02; H, 6.27; N, 11.37%. found: C, 78.03; H, 6.27; N, 11.40%.

### Example 17: [4-(4-isopropyl-phenyl)-phthalazin-1-yl]-naphthalen-2-yl-amine (15)

Beige powder; mp.:180-183° C.; FT-IR (KBr, ν, cm⁻¹): 2958, 2878 (—CH, —CH₃), 3071-3030 (═CH, Ar), 1609, 1459 (C═C), 3450 (—NH), 1188 (—C—N); ¹H NMR, δ (ppm) in DMSO-d₆: 1.28 (d, 6H, 2CH₃), 3.45 (m, 1H, —CH(CH₃)₂), 6.76-8.01 (m, 15H, —CH—, ArH), 10.57 (br, 1H, —NH—); ¹³C NMR, δ (ppm) in DMSO-d₆: 24.3 (2C, —CH₃), 31.7 (1C, —CH(CH₃)₂), 115.4, 119.0, 126.7, 127.4, 133.1, 148.1 (19C, ═CH—, Ar), 117.1, 127.3, 127.1 (3C, ═CH—, pyridazine), 142.6, 166.6 (2C, ═C—NH—C═); MS (m/z): 389.19 (M⁺, 100.0), 390.19 (M⁺+1, 27.1); Anal. calcd. for C₂₇H₂₃N₃/(389.49): C, 83.26; H, 5.95; N, 10.79%. found: C, 83.21; H, 5.98; N, 10.80%.

### Example 18: 5-(4-isopropyl-phenyl)-6,6a,12-triaza-benzo[a]anthracen-7-one (16)

White powder; mp.: 190-191° C.; FT-IR (KBr, ν, cm⁻¹): 2957, 2862 (—CH, —CH₃), 3060-3010 (═CH, Ar), 1600, 1475 (C═C), 1670 (C═O); ¹H NMR, δ (ppm) in DMSO-d₆: 1.20 (d, 6H, 2CH₃), 3.42 (m, 1H, —CH(CH₃)₂), 7.2-7.5 (m, 12H, —CH—, ArH); ¹³C NMR, δ (ppm) in DMSO-d₆: 24.4 (2C, —CH₃), 31.7 (1C, —CH(CH₃)₂), 110, 116, 126.4, 128.4, 131, 133 (16C, ═CH—, Ar), 134.3, 155.6 (2C, ═CH, pyridazine), 151, 169 (2C, —C5N); 170 (1C, —C50); MS (m/z): 365.15 (M⁺, 100), 366.16 (M⁺+1, 27.0) Anal. calcd. for C₂₄H₁₉N₃O (365.43): C, 78.88; H, 5.24; N, 11.50%. found: C, 78.90; H, 5.26; N, 11.52%.

### Example 19: [4-(4-isopropyl-phenyl)-phthalazin-1-yl]-diphenyl-amine (17)

White powder; mp.:203-204° C.; FT-IR (KBr, ν, cm⁻¹): 2962, 2852 (—CH, —CH₃), 3070-3010 (═CH, Ar), 1600, 1475 (C═C), 1360 (—C—N); ¹H NMR, δ (ppm) in DMSO-d₆: 1.25 (d, 6H, 2CH₃), 3.43 (m, 1H, —CH(CH₃)₂), 7.35-8.07 (m, 18H, —CH—, ArH), ¹³C NMR, δ (ppm) in DMSO-d₆: 24.3 (2C, —CH₃), 33.7 (1C, —CH(CH₃)₂), 117, 120, 125.6, 127, 128.8, 129.6, 133.1, 147.1 (20C, ═CH—, Ar), 131, 133.3, 151.1 (3C, ═CH—, pyridazine), 143, 169 (3C, —N—C═); MS (m/z): 415.20 (M⁺, 100.0), 416.21 (M⁺+1, 15.1); Anal. calcd. for C₂₉H₂₅N₃/(415.53): C, 83.82; H, 6.06; N, 10.11%. found: C, 83.84; H, 6.04; N, 10.09%.

### Example 20: Synthesis of Compounds Pyrazole Phthalazine Derivatives (18-21)

A mixture (2.84 g, 10 mmol) of 2 (10 mmol) of acetyl acetone (1.00 mL), and/or ethyl acetoacetate (1.27 mL), ethyl cyanoacetate (1.13 mL), and malononitrile (0.55 g) was dissolved in 30 mL ethanol with C₂H₅ONa, then refluxed in an oil bath for 5 h at 80° C. (MW, 10 min). After an excess solvent is distilled, off, poured into cold water, then extracted with diethyl ether. The solid crystallized from benzene, then dis solved in 30 mL of ethanol, and (0.9 mL, 20 mmol) of hydrazine hydrate, and refluxed to give 18-21. The separated solid crystallized from a suitable solvent.

### Example 21: 1-(3,5-Dimethyl-4H-pyrazol-4-(4-isopropyl-phenyl)-phthalazine (18)

White crystals; mp.:270-271° C.; FT-IR (KBr, ν, cm⁻¹): 2962,2852 (—CH, —CH₃), 3060, 3010 (═CH, Ar), 1598, 1440 (C═C), 1610 (C—N—N); ¹H NMR, δ (ppm) in DMSO-d₆: 1.29 (d, 6H, 2-CH₃), 2.00 (s, 6H, 2-CH₃, pyrazole), 2.50 (d, 1H, —CH—, pyrazole), 3.12 (m, 1H, —CH(CH₃)₂), 7.4-8.3 (m, 8H, —CH—, ArH); ¹³C NMR, δ (ppm) in DMSO-d₆: 18.2 (2C, —CH₃, pyrazole), 24.3 (2C, —CH₃), 33.7 (1C, —CH(CH₃)₂), 39.9 (1C, —CH—, pyrazole), 126.5, 127.1, 128.7, 129 (1° C., ═CH—, Ar), 132, 134.5, 150.6 (4C, ═CH—, pyridazine), 160 (2C, —C—CH₃, pyrazole); MS (m/z): 342.18 (M⁺, 100.0), 341.19 (M⁺−1, 24.8); Anal. calcd. for C₂₂H₂₂N₄/(342.44): C, 77.16; H, 6.48; N, 16.36%. found: C, 77.18; H, 6.45; N, 16.38%.

### Example 22: 4-[4-(4-isopropyl-phenyl)-phthalazin-1-yl]-5-methyl-2,4-dihydro-pyrazol-3-one (19)

White crystal; mp.:250-252° C.; FT-IR (KBr, ν, cm⁻¹): 2963,2853 (—CH, —CH₃), 3067-3020 (═CH, Ar), 1598,1440 (C═C), 1680 (C═O), 1610 (C—N—N); ¹H NMR, δ (ppm) in DMSO-d₆: 1.29 (d, 6H, 2CH₃), 2.00 (s, 3H, —CH₃), 3.43 (m, 1H, —CH(CH₃)₂), 5.21 (s, 1H, —CH—, pyrazole), 7.243-8.3 (m, 8H, —CH—, ArH), 12.85 (br, 1H, —NH); ¹³C NMR, δ (ppm) in DMSO-d₆: 15.3 (1C, —CH₃), 24.3 (2C, —CH₃), 33.8 (1C, —CH(CH₃)₂), 40.3 (1C, —CH—, pyrazole), 126.6, 127, 128.3, 129.7, 134 (1° C., ═CH—, Ar), 132, 147, 150 (4C, ═CH—, pyridazine), 155.6 (1C, —CH—, pyrazole), 159.7 (1C, —C50); MS (m/z): 344.16 (M⁺, 100.0); 345.17 (M⁺+1, 20); Anal. calcd. for C₂₁H₂₀N₄O/(344.41): C, 73.23; H, 5.85; N, 16.27%. found: C, 73.20; H, 5.86; N, 16.27%.

### Example 23: 5-Amino-4-[4-(4-isopropyl-phenyl)-phthala zin-1-yl]-2,4-dihydro-pyrazol-3-one (20)

Light brown crystals; mp.:169-170° C.; FT-IR (KBr, ν, cm⁻¹): 2962,2850 (—CH, —CH₃), 3065-3025 (═CH, Ar), 1625,1475 (C═C), 1682 (C═O), 3409, 3350 (—NH₂), 1625 (C—N—N); ¹H NMR, δ (ppm) in DMSO-d₆: 1.28 (d, 6H, 2CH₃), 3.01 (m, 1H, —CH(CH₃)₂), 4.7 (s, 1H, —CH—, pyrazole), 7.43-8.34 (m, 8H, —CH—, ArH), 12.85, 11.9 (br, 3H, —NH₂, —NH—); ¹³C NMR, δ (ppm) in DMSO-d₆: 24.3 (2C, —CH₃), 33.8 (1C, —CH(CH₃)₂), 51.0 (1C, —CH—, pyrazole), 126.5, 127, 128.3, 128.7, 129 (1° C., ═CH—, Ar); 132.6, 134.5, 155.6 (4C, ═CH—, pyridazine), 149.7 (1C, ═C—NH₂, pyrazole), 159.7 (1C, —C50); MS (m/z): 345.16 (M⁺, 51.39), 347.17 (M⁺+2, 31.0); Anal. calcd. for C₂₀H₁₉N₅O/(345.40):C, 69.55; H, 5.54; N, 20.28%. found: C, 69.57; H, 5.55; N, 20.29% b.

### Example 24: 4-[4-(4-isopropyl-phenyl)-phthalazin-1-yl]-4H-pyrazole-3,5-diamine (21)

Light brown powder; mp.:172-173° C.; FT-IR (KBr, ν, cm⁻¹): 2960,2856 (—CH, —CH₃), 3070-3030 (═CH, Ar), 1600,1465 (C═C), 3450,3300 (—NH₂), 1625 (C—N—N); ¹H NMR, δ (ppm) in DMSO-d₆: 1.28 (d, 6H, 2CH₃), 3.42 (m, 1H, —CH(CH₃)₂), 2.65 (d, 1H, —CH—, pyrazole), 7.36-8.27 (m, 8H, —CH—, ArH), 12.85, 11.87 (br, 4H, —NH₂); ¹³C NMR, δ (ppm) in DMSO-d₆: 24.4 (2C, —CH₃), 33.8 (1C, —CH(CH₃)₂), 40.3 (1C, —CH—, pyrazole), 126.5, 127.1, 128.4, 129, 146 (1° C., ═CH—, Ar), 132.1, 134.1, 150 (4C, ═CH—, pyridazine), 160 (2C, ═CH—NH₂); MS (m/z): 344.17 (M⁺, 100.0), 345.18 (M⁺+1, 22.5); Anal. calcd. for C₂₀H₂₀N₆ (344.41): C, 69.75; H, 5.85; N, 24.40%. found: C, 69.77; H, 5.88; N, 24.38%.

### Example 25: Cell Culture

In the present disclosure, three cancer cell lines were utilized, each with a maximum of 15 passages. These cell lines represent three different organs: the breast (Michigan Cancer Foundation-7 (MCF7)), colon (Human Colon Tumor 116 (HCT116)), and liver (Human Hepatocellular Carcinoma G2 (HepG2)). To assess the selectivity of the compounds, a normal fetal lung fibroblast cell line, Medical Research Council cell strain 5 (MRC5), was also used, with approximately 10 passages. All cells were sourced from the American Type Culture Collection (ATCC) in the United States. The three cancer cell lines were maintained in RPMI-1640 media supplemented with 10% fetal bovine serum (FBS), while MRC5 cells were maintained in Eagle's Minimum Essential Medium (10% FBS), under standard conditions of 37° C., 5% carbon dioxide, and 100% relative humidity.

### Example 26: Anticancer and Selectivity Studies

To evaluate the anticancer activity of an investigational compound, 19 compounds, along with doxorubicin, were tested using the 3-(4,5-dimethylthiazol-2-yl)-2,5-diphenyltetrazolium bromide (MTT) assay, following a method from a previous study (See: Abdalla, A. N., Qattan, A., Malki, W. H., Shahid, I., Hossain, M. A., & Ahmed, M. (2020). Significance of targeting VEGFR-2 and Cyclin D1 in Luminal—A breast cancer. Molecules, 25(20), 4606, incorporated herein by reference in its entirety). Each cell line was subcultured until it reached 80%-90% confluency before treatment with each compound or doxorubicin at final concentrations ranging from 0 to 50 millimolar (mM) for 72 h. Afterward, MTT was added to each well, including negative controls, and the cells were incubated for 3 h at 37° C. The MTT solution was then removed, and the resulting precipitation was dissolved in dimethyl sulfoxide (DMSO). The absorbance at 550 nm (A550) for each well and concentration was measured using a multi-plate reader (Bio-Rad, PR 4100, Hercules, CA, USA), and the half-maximal inhibitory concentration (IC₅₀) was determined using GraphPad Prism software (version 5). The selectivity index (SI) for each of the 19 compounds and doxorubicin was calculated by dividing the IC₅₀ against MRC-5 cells by the IC₅₀ against either MCF7, HCT116, or HepG2 cells.

### Example 27: Molecular Modeling

Molecular docking simulations and ligand binding energy calculations were carried out using the Molecular Operating Environment (MOE) 2014 (CCGV®, Montreal, Canada). Visualization of binding interactions and figure generation were performed using PyMol 2.0.6 Graphical Software (Schrödinger®, New York, NY, USA). Ligand preparation, energy minimization, and potential energy calculations were conducted using the MOE interface. The crystal structure of human Poly (ADP-ribose) polymerase 1 (PARP-1) (PDB code: 7AAD), co-crystallized with Olaparib, was chosen for the docking studies. The target protein and ligands were prepared using the protein preparation and LigX tools, respectively. The protein underwent three-dimensional protonation, auto-correction of atom types and connections, and partial charge assignment at a physiological neutral pH of 7.4. The ligand PDB file was modified by adding hydrogens, calculating partial charges, and adjusting the ionization state. Energy minimization was performed using the MMFF94X force field. The active site for docking was defined by residues within 5 angstroms of the co-crystallized ligand. The docking procedure was performed using the MOE docking tool within the MOE software, utilizing the default parameters. This process employed rigid docking, where the protein remained fixed, while the ligands were kept flexible, and the ligand's conformation was generated through ligand placement and bond rotation. The docking output was set to provide 10 docking poses per ligand. The results were analyzed by identifying potential ligand interactions with the protein's active site using the MOE-ligand interactions wizard and bond distance calculations in PyMol. The binding score (S score) in MOE, along with comparative docking against the crystal ligand, were also used to identify the best binders. The selection of the most favorable ligands was based on a comprehensive approach that considered the best docking scores, low root mean square deviation (RMSD) values compared to the co-crystallized ligand, and key interactions with the active site.

### Results and Discussion

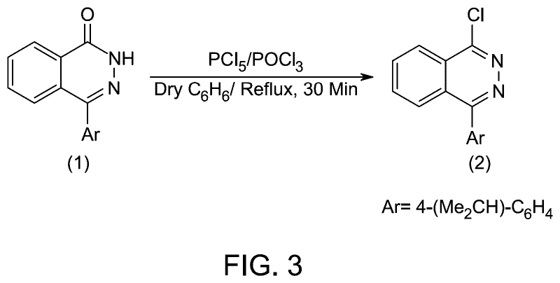

Phthalazine and phthalazinone derivatives have garnered significant attention in medicinal chemistry and drug discovery and had been reported to have a wide range of biological actions and medical applications such as antimicrobial, anticancer, antitumor, anti-Alzheimer's inhibitors, anti of acquired immunodeficiency syndrome (anti-AIDS), anti-inflammatory, antiproliferative agents, anticonvulsant, and antidiabetic. In addition, phthalazinone 1 was synthesized in a prior study conducted by Abubshait and researchers (See: Abubshait, S. A., Abubshait, H. A., Almalih, R., Gomaa, M. S., Nawaz. M., Ababutain, I. M., & Alghamdi, A. 1. (2022). Microwave-assisted synthesis of phthalazinone derivatives with biological activity and in silico antiproliferative studies. ChemistrySelect, 7(46), e202203925, incorporated herein by reference in its entirety). Chlorophthalazine 2 was produced by refluxing compound 1 with a combination of phosphorus pentachloride and phosphorus oxychloride and FIG. 3 shows the corresponding synthesis scheme.

The structure of chlorophthalazine for FT-IR spectra showed conforming stretching bands for aliphatic and aromatic groups, with major bands at 2922 cm⁻¹, 2865 cm⁻¹ (—CH, —CH₃), 1635 cm⁻¹, 1448 cm⁻¹ (C—H, C—H₃), 3068-3010 cm⁻¹ (C═CH, Ar), and C═C, along with the disappearance of the carbonyl group of phthalazinone. Moreover, the ¹³C NMR spectra of compound 2 showed the presence of obvious bands for —CH₃, —CH(CH₃), related to pyridazine, and aromatic groups. New protons and carbon bands were observed at 1.28 (d, 6H, 2CH₃), 2.98 (m, 1H, —CH(CH₃)₂), 30.9 (1C, —CH(CH₃)), 126.7, 127.5, 128.5, 132.0, 148.0 (10C, ═CH, Ar), 130.6, 134.0, 151.6 (3C, ═CH, pyridazine), and 171.0 (1C, —C-Cl).

The one-pot reaction of chlorophthalazine is an important synthetic application for producing a heterocyclic nucleus with broad pharmaceutical and biological activity. This study investigates the nucleophilic displacement reactions of chlorophthalazine with different hydrazine derivatives, primary and secondary amines, and active methylene compounds to produce novel phthalazine derivatives. The reactions were conducted using both traditional methods and microwave irradiation to synthesize new phthalazine derivatives from compound 2 with high yields and a rapid reaction rate. Microwave irradiation was employed as a green and efficient technology to assist in the synthesis process.

The use of microwave irradiation in the present synthesis of heterocyclic compounds offers several advantages over traditional methods. It accelerates reaction rates, improves yields, and enhances the selectivity of various organic transformations. In contrast, traditional synthetic methods for heterocycles often rely on multi-step processes that involve distinct reaction steps and purification procedures. These conventional methods typically encounter drawbacks, such as decreased productivity, extended reaction times, and the production of by-products. However, using microwave irradiation as a green strategy for the improved synthesis of biologically and pharmaceutically interesting annulated heterocycles has further highlighted the potential of this approach in accessing novel heterocyclic systems.

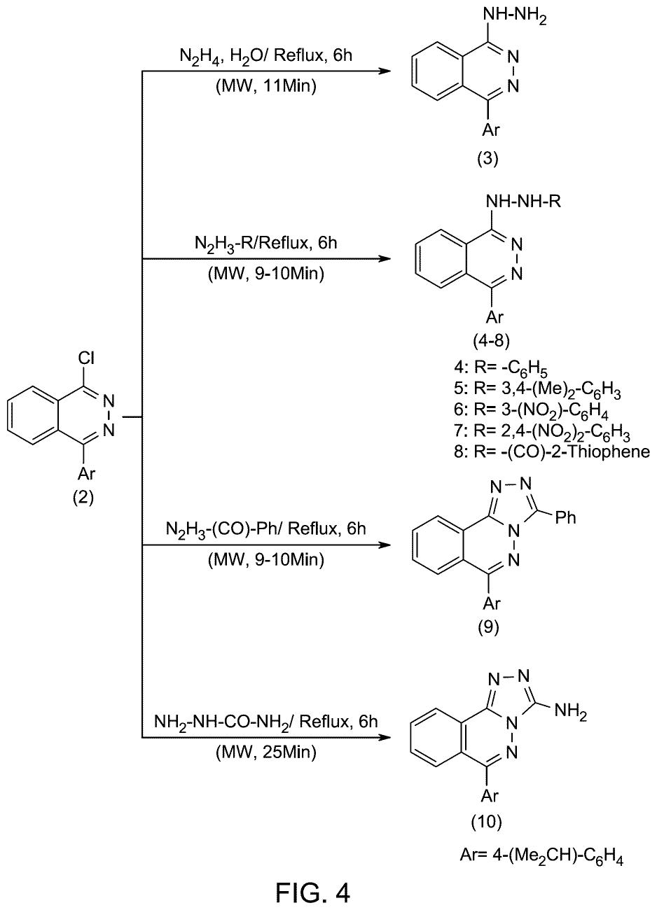

FIG. 4 illustrates the reaction of chlorophthalazine (compound 2) with hydrazine hydrate, phenylhydrazine, 3,4-dimethylphenylhydrazine, 3-nitrophenylhydrazine, 2,4-dinitrophenylhydrazine, 2-thiophenecarboxylic hydrazine, benzoylhydrazine, and semicarbazide, which were experimented under reflux for 5-6 h and under microwave irradiation for 9-25 min in dry benzene to yield compounds 3-10, respectively. The FT-IR spectra of the hydrazine derivatives showed new (—NHNH₂, —NHNH—) stretching bands in the regions 3421-3304 cm⁻¹ and 3319-3210 cm⁻¹.

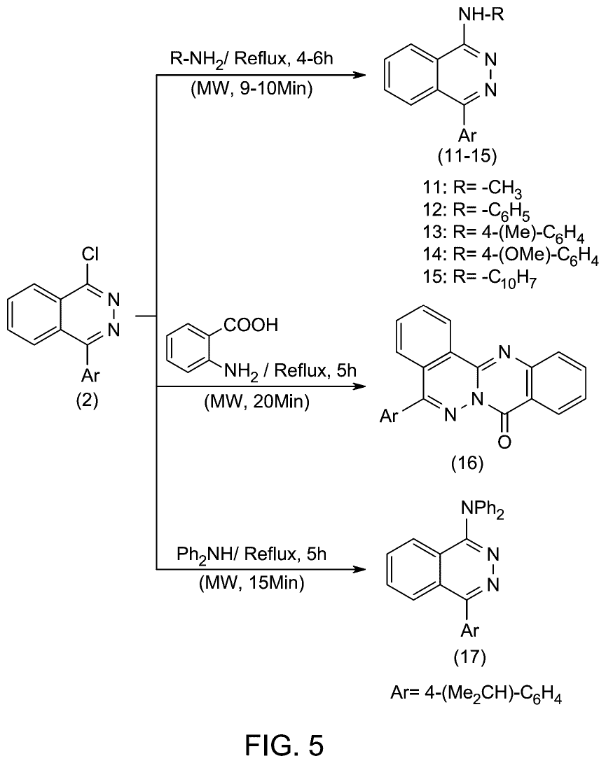

The displacement of chloride from chlorophthalazine can be achieved through nucleophilic attacks with electron-rich groups, specifically primary and secondary amines such as methylamine, aniline, p-toluidine, p-anisidine, a-naphthylamine, anthranilic acid, and diphenylamine. This can be accomplished via reflux for 4-6 h or microwave irradiation for 9-10 min to yield products 11-17, respectively. FIG. 5 shows this reaction. Compounds 11-17 exhibited bands at 3450 cm⁻¹ and 1188-1360 cm⁻¹, which can be attributed to the presence of secondary and tertiary amine groups (—NH, —C—N).

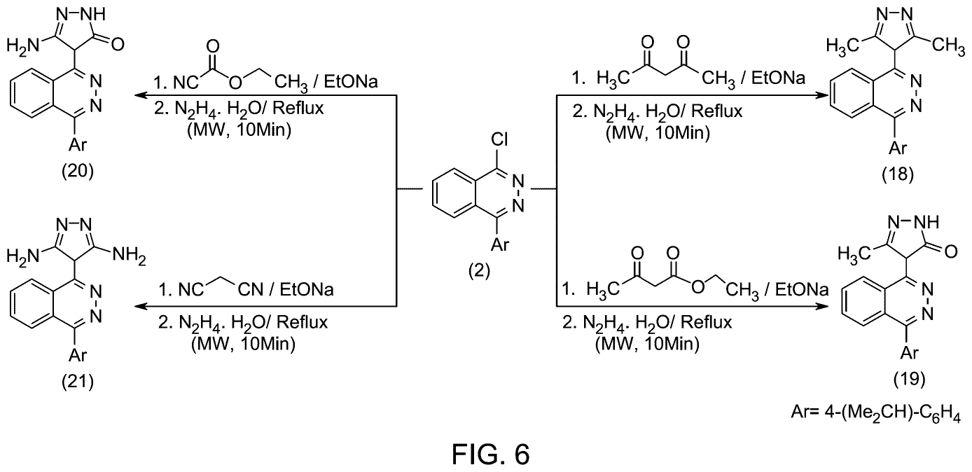

The reaction between compound 2 and carbon nucleophiles, involving active methylene compounds, resulted in the formation of pyrazolo derivatives. Phthalazine derivatives were obtained by reacting compound 2 with acetylacetone, ethyl acetoacetate, ethyl cyanoacetate, and malononitrile in the presence of sodium ethoxide. These derivatives were further subjected to a cyclization process with hydrazine hydrate in ethanol, leading to the formation of pyrazolo derivatives 18-21, respectively, as shown in FIG. 6.

Coupling of compound 2 with various active methylene compounds produced the corresponding coupling products using conventional methods in ethanol under reflux for 6 h. In comparison, the reaction was successfully performed within a shorter time frame under microwave irradiation. In addition to a shorter reaction time, the products were obtained in higher yields compared to the conventional method. FIG. 6 represents the pathway for preparing the target compounds through the nucleophilic attack of compound 2 with active methylene compounds using microwave irradiation. The synthesized compounds underwent a cyclization process due to the nucleophilic attack by hydrazine hydrate, resulting in the formation of the corresponding pyrazolyl derivatives.

A comprehensive analysis of the unique products was conducted using their spectral data. The IR spectra exhibited absorption bands in the range of 1680 cm⁻¹, which can be attributed to the (C═O) functional group associated with compounds 19 and 20. Compounds 20 and 21 exhibited new stretching bands at positions 3409-3450-3350-3000 cm⁻¹, which can be attributed to the (NH₂) group. The ¹H NMR spectra exhibited prominent bands corresponding to isopropyl, aliphatic, and aromatic protons. Additionally, novel bands were observed for methyl and pyrazole protons at a chemical shift of 2.00 and 2.50 ppm, respectively. The spectra of compounds 18-21 also showed new signals for the (NH₂, —NH—) group. Table 1 presents the yield and reaction time (in mm) for compounds 2-21, obtained using microwave irradiation and the conventional approach.

TABLE 1
Yield (%) and reaction time (min) of compounds 2-21 by
using microwave irradiation and conventional method.

| | Reaction time (min) | | | |
|---|---|---|---|---|
| Com- | Conven- | | Yield (%) | |
| pound | tional | Microwave | Conventional | Microwave |
| No. | methods | irradiation | methods | irradiation |
| 2 | 30 | — | (1.67 mg) 60.0% | — |
| 3 | 360 | 11 | (2.39 mg) 86.0% | (2.73 mg) 98.0% |
| 4 | 360 | 10 | (2.73 mg) 77.0% | (3.47 mg) 98.0% |
| 5 | 360 | 10 | (2.62 mg) 68.4% | (3.72 mg) 97.0% |
| 6 | 360 | 10 | (3.03 mg) 76.0% | (3.94 mg) 98.8% |
| 7 | 360 | 9 | (3.33 mg) 75.0% | (3.93 mg) 88.6% |
| 8 | 360 | 9 | (3.34 mg) 76.1% | (4.21 mg) 95.0% |
| 9 | 360 | 9 | (3.25 mg) 70.0% | (4.17 mg) 89.8% |
| 10 | 360 | 25 | (2.42 mg) 80.1% | (2.79 mg) 92.2% |
| 11 | 300 | 9 | (2.11 mg) 76.3% | (2.35 mg) 85.0% |
| 12 | 300 | 9 | (2.71 mg) 80.0% | (3.29 mg) 97.0% |
| 13 | 240 | 10 | (2.71 mg) 76.9% | (3.42 mg) 97.0% |
| 14 | 360 | 10 | (3.23 mg) 87.5% | (3.41 mg) 92.3% |
| 15 | 300 | 9 | (3.00 mg) 77.2% | (3.27 mg) 84.0% |
| 16 | 300 | 20 | (3.07 mg) 84.0% | (3.58 mg) 98.0% |
| 17 | 300 | 15 | (1.94 mg) 46.7% | (4.03 mg) 97.0% |
| 18 | 300 | 10 | (2.10 mg) 61.3% | (2.99 mg) 87.3% |
| 19 | 300 | 10 | (1.89 mg) 55.0% | (2.58 mg) 75.8% |
| 20 | 300 | 10 | (1.73 mg) 50.0% | (2.74 mg) 79.5% |
| 21 | 300 | 10 | (1.98 mg) 57.5% | (2.94 mg) 85.5% |

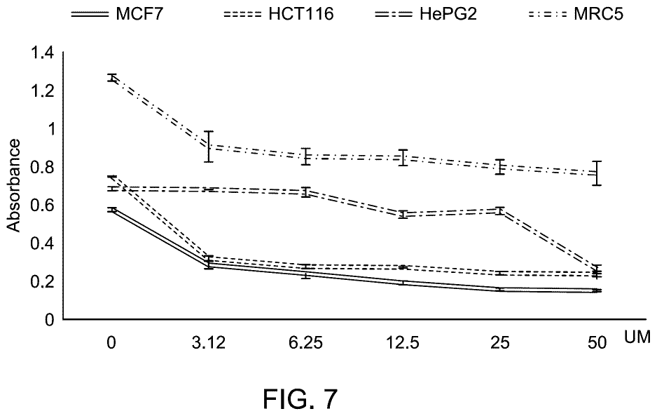

To determine the anticancer effect of compounds 3-21 against MCF7, HCT116, and HepG2 cancer cells, an MTT assay was performed. The most active compound against MCF7 cells was compound 13, with an IC₅₀ of 0.286 micromolar (μM), which was less effective but comparable to the IC₅₀ of doxorubicin against MCF7 (0.041 μM). The least active compound was compound 4, with an IC₅₀ of 5.370 μM. The range of anticancer activity against HCT116 cells was wider, with compound 6 showing an IC₅₀ of 0.384 μM, which was 10 times more effective than the anticancer effect of doxorubicin against HCT116 (IC₅₀ 3.964 μM). In contrast, compound 3 was less active against HCT116, with an IC₅₀ of 25.228 μM. Regarding HepG2 cells, the most active compound was compound 16, with an IC₅₀ of 0.484 μM, which was comparable to the IC₅₀ of doxorubicin against HepG2 cells (0.381 μM). The least active compound was compound 4, with an IC₅₀ of 5.808 μM. Therefore, the most active compounds were 13, 6, and 16, respectively (Table 2). Table 2 also includes the anticancer effect of each compound against MRC5 normal fibroblast cells. The IC₅₀ ranges between 0.839 mM and 25.800 mM. This data was used to calculate the SI of each compound towards MRC5 normal fibroblast cells (Table 3) compared with each of the three cell lines, and the best SI was for compound 6 (FIG. 7), followed by compound 13, all of which was comparable with the selectivity of doxorubicin. The best selective compounds were compound 6 selective against three cells compared to MRC5 cells, followed by compounds 13, 16, 8, and 14, best cytotoxic and selective compounds 6 and 13 (Table 3).

TABLE 2
Anticancer activity of compounds 3-21 and doxorubicin against three cell
lines, and MRC5 normal fibroblast cells (MTT 72 h, IC₅₀ “mM” ± SD, n = 3).

| | | | | cytotoxicity* | |
|---|---|---|---|---|---|
| Compound No. | MCF7 | HCT116 | HepG2 | (IC₅₀) | MRC5 |
| 3 | 1.965 ± 0.393 | 25.228 ± 2.713 | 4.715 ± 1.098 | 10.636 | 12.145 ± 0.205 |
| 4 | 5.370 ± 0.282 | 1.954 ± 1.310 | 5.808 ± 0.997 | 4.377 | 19.270 ± 1.456 |
| 5 | 2.881 ± 0.844 | 27.126 ± 1.447 | 2.978 ± 0.169 | 10.995 | 7.894 ± 0.474 |
| 6 | 1.739 ± 0.295 | 0.384 ± 0.045 | 1.52 ± 0.025 | 1.214 | 25.800 ± 1.004 |
| 7 | 2.578 ± 0.074 | 5.087 ± 0.369 | 3.668 ± 0.804 | 3.777 | 20.935 ± 1.152 |
| 8 | 1.255 ± 0.188 | 16.592 ± 3.157 | 4.277 ± 1.324 | 7.375 | 16.885 ± 0.629 |
| 9 | 1.485 ± 0.363 | 17.389 ± 0.310 | 2.031 ± 0.033 | 6.968 | 8.808 ± 0.596 |
| 10 | 5.097 ± 0.014 | 2.584 ± 0.443 | 4.162 ± 0.415 | 3.947 | 3.168 ± 0.237 |
| 11 | 1.313 ± 0.231 | 4.160 ± 0.691 | 4.336 ± 0.796 | 3.27 | 11.030 ± 1.173 |
| 12 | 1.390 ± 0.468 | 5.358 ± 0.364 | 5.065 ± 1.044 | 3.937 | 6.077 ± 0.343 |
| 13 | 0.286 ± 0.038 | 4.846 ± 0.101 | 1.006 ± 0.143 | 2.046 | 11.620 ± 1.527 |
| 14 | 0.988 ± 0.067 | 14.031 ± 1.143 | 2.110 ± 0.580 | 5.71 | 11.555 ± 0.756 |
| 15 | 0.843 ± 0.752 | 5.022 ± 0.472 | 3.367 ± 0.364 | 3.077 | 6.288 ± 0.581 |
| 16 | 1.702 ± 0.649 | 10.992 ± 0.682 | 0.484 ± 0.377 | 4.393 | 7.929 ± 0.382 |
| 17 | 1.460 ± 0.235 | 13.884 ± 2.268 | 1.550 ± 0.264 | 5.631 | 13.050 ± 0.862 |
| 18 | 1.456 ± 0.295 | 12.255 ± 1.887 | 2.179 ± 0.774 | 5.296 | 5.205 ± 0.840 |
| 19 | 1.056 ± 0.027 | 2.619 ± 1.429 | 1.735 ± 0.333 | 1.803 | 9.023 ± 0.603 |
| 20 | 1.625 ± 0.259 | 1.780 ± 0.710 | 1.624 ± 0.327 | 1.676 | 4.896 ± 0.571 |
| 21 | 1.512 ± 0.497 | 5.728 ± 1.571 | 3.136 ± 0.189 | 3.459 | 0.839 ± 0.084 |
| Doxorubicin | 0.041 ± 0.002 | 3.964 ± 0.360 | 0.381 ± 0.133 | 1.462 | 2.476 ± 0.033 |

*Average cytotoxicity (IC₅₀) of each compound against the three cancer cells. When SI is more than 1 = selectivity.

TABLE 3
SI of compounds 3-21* and doxorubicin
against MRC5 normal fibroblast cells.

| Compound No. | MCF7 | HCT116 | HepG2 |
|---|---|---|---|
| 3 | 0.57 | 0.48 | 2.57 |
| 4 | 3.62 | 9.88 | 3.31 |
| 5 | 2.73 | 0.29 | 2.65 |
| 6 | 15.17 | 67.89 | 16.97 |
| 7 | 8.37 | 4.12 | 5.71 |
| 8 | 14.06 | 1.01 | 3.95 |
| 9 | 6.28 | 0.5 | 4.33 |
| 10 | 0.62 | 1.22 | 0.75 |
| 11 | 8.41 | 2.65 | 2.54 |
| 12 | 4.36 | 1.13 | 1.19 |
| 13 | 41.5 | 2.39 | 11.62 |
| 14 | 11.78 | 0.82 | 5.47 |
| 15 | 4.51 | 1.25 | 1.86 |
| 16 | 4.65 | 0.72 | 16.5 |
| 17 | 8.93 | 0.93 | 8.41 |
| 18 | 3.58 | 0.42 | 2.39 |
| 19 | 8.59 | 3.45 | 5.21 |
| 20 | 3.01 | 2.74 | 3.01 |
| 21 | 0.54 | 0.14 | 0.26 |
| Doxorubicin | 60.39 | 0.62 | 6.48 |

*A compound is considered selective when SI ≥ 1.

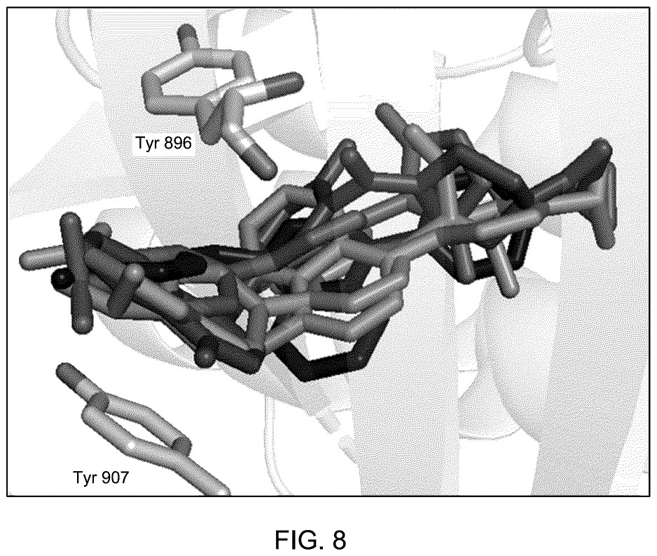

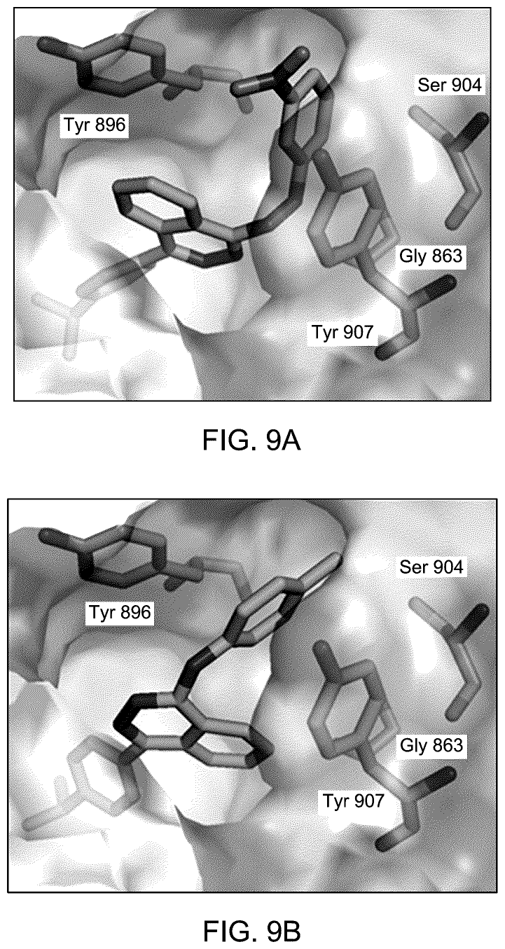

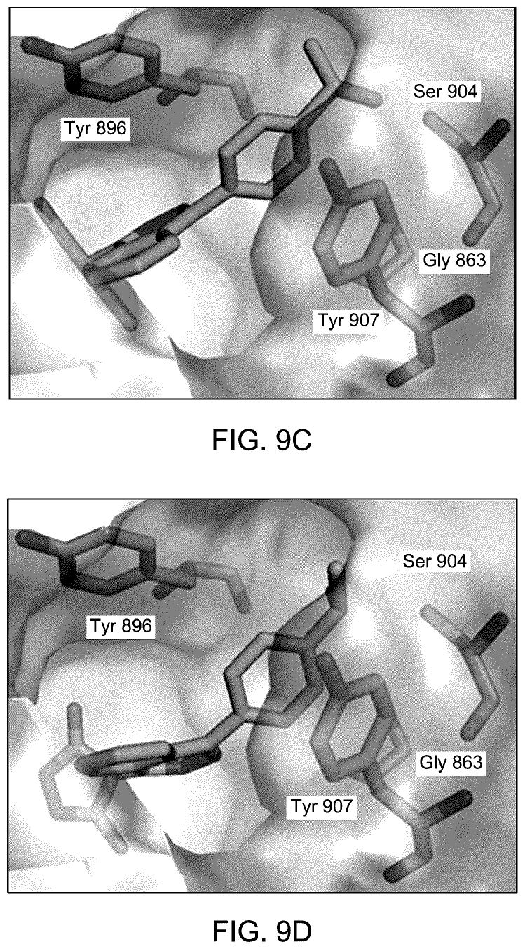

Molecular docking simulations performed on the synthesized compounds with poly ADP-ribose polymerase (PAPR-1) revealed a favorable binding affinity. FIG. 8 shows a schematic for comparative binding positions of compound 6, compound 13, compound 19, compound 20, and olaparib bound to poly (ADP-Ribose) polymerase 1 (PARP-1) (7aad) active site. The results confirmed the experimental IC₅₀, which predominantly fell within the one-digit micro molar range, and successfully identified the compounds with the highest experimental activity (6, 13, 19, 20) as the best binders. Those compounds were especially aligned with the standard co-crystallized inhibitor, Olaparib in the active site (FIG. 8). The docking studies showed that the most active compounds were able to bind the gatekeeper residues, Tyr 896, and Tyr 907, which were identified in our previous study as being crucial for enzyme binding. The initial binding with these two residues is the starting point for ligands to access the active site, and the p-p interactions formed with these residues give affinity and selectivity to the interacting ligand. FIG. 9A-9D shows a schematic for surface representation of compound 6, 13, 19, and 20 bound to PARP-1 (7aad) active site. In the most active compounds, one of the aromatics interacts with Tyr 907 while the phthalazinone ring performs the interaction with Tyr 896. The less lipophilic phthalazinone ring is the part of the structure that is solvent-exposed, while the more lipophilic isopropyl-phenyl group at position 4 is directed deeper into the binding pocket, stabilizing the compounds within the active site. This stabilization occurs either in the small binding pocket of the enzyme formed by Gly 863, Ser 907, and Glu 988, as seen with compounds 19 and 20 (FIG. 9C-9D), or in the more spacious hydrophobic pocket formed by Tyr 710, Asp 766, Leu 769, Arg 878, Ala 880, Pro 881, and Tyr 889, as seen with compounds 6 and 13 (FIG. 9A-9B).

The substituted nitrogen moiety at position 1 occupies the opposite side of the binding pocket. This interaction was particularly observed for compounds 6 and 13, likely due to the higher flexibility of the anilino or phenylhydrazino moiety compared to the more rigid pyrazole ring in compounds 19 and 20. This increased flexibility allows compounds 6 and 13 to fit better in the smaller pocket.

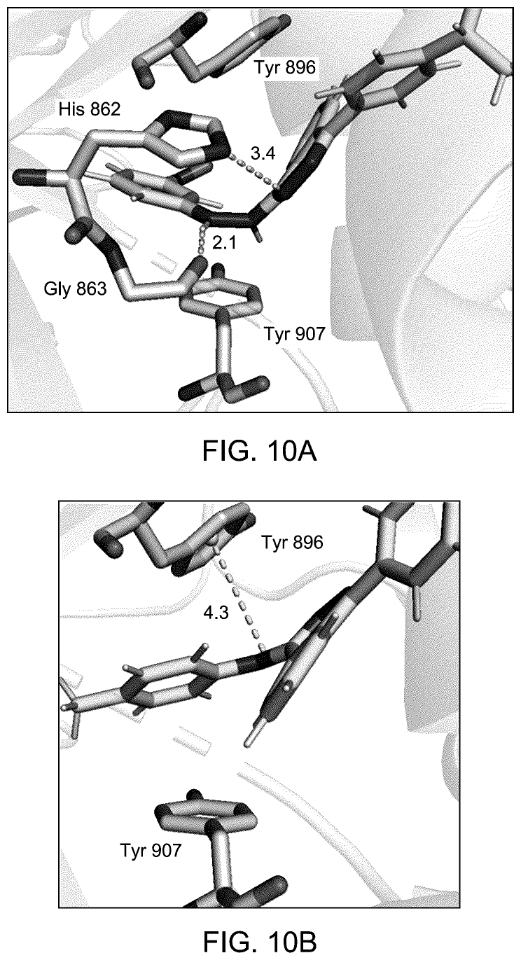

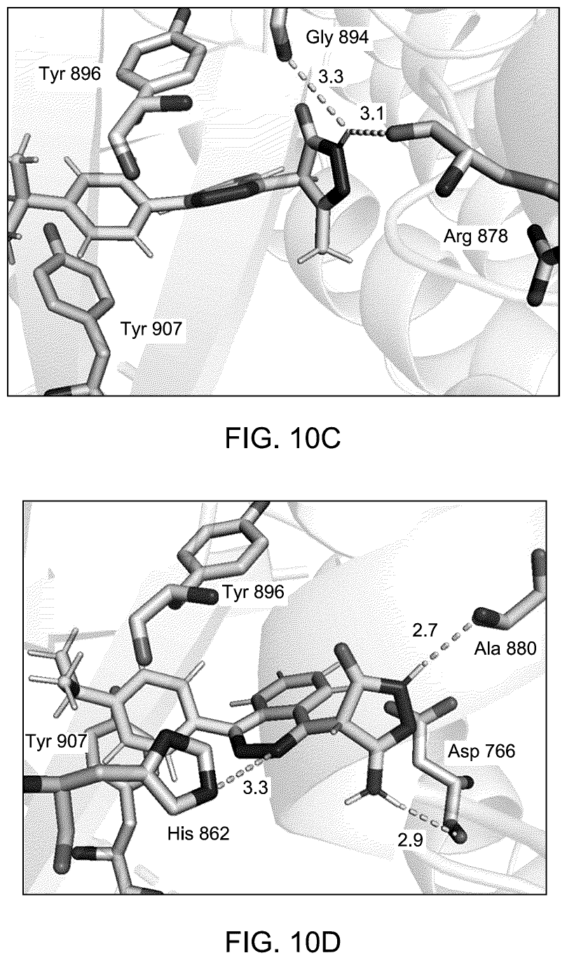

Compound 6 (IC₅₀, 1.214) established two additional electrostatic interactions with its phthalazinone and side chain hydrazino nitrogens and His 862 sidechain and Gly 863 backbone, respectively (FIG. 10A). Compound 13 (IC₅₀, 2.046) did not show additional electrostatic bonds, however, a potential and characteristic p-H bonding was noted with its anilino nitrogen and Tyr 896 which may explain the comparable activity with the other compounds despite the lack of direct H-bonding (FIG. 10B). Compound 19 (IC₅₀, 1.803) was able to gain high affinity through H-bonding with its pyrazole ring and Arg 878 and Gly 894 backbones, where it acted as a strong H-bond donor (FIG. 10C). Compound 20 (IC₅₀, 1.676) was identified as one of the best binder due to formation of several H-bonds with residues in the binding pocket more specifically with its pyrazole and Ala 880 backbone and its amine and Asp 766 backbone and its phthalazinone nitrogen and His 862 sidechain (FIG. 10D). The results of the biological testing and the docking studies formulated a primary structure-activity relationship for the phthalazine derivatives. The phthalazine core is important and is involved in anchoring the compounds at the active site. Substitution at position 1 with a phenyl ring gives more active compounds as the aromatic ring digs deeper in the active site and establishes hydrophobic interactions and p-p stacking. Substitution at position 4 with amino functionality is important for the activity, and it can accommodate aminophenyl, hydrazinophenyl, and aminoheterocycles as examples. However, increasing the steric hindrance in this part such as disubstituted amine (compound 17) or ring expansion (compound 16), reduced the activity. The fact that PARP is overexpressed in the tested cancer cells together with the high affinity of the synthesized compounds, imparted a high selectivity on cancer cells compared to normal cells with compounds 6, 8, 13, 14, and 16 being the most selective. Compounds 6 and 13 especially showed selectivity comparable to the clinically used drug doxorubicin in MCF7 cells (15.17 and 41.5, respectively, compared to 60.39) and superior selectivity on HCT116 (67.89 and 2.39, respectively, compared to 0.62) and HepG2 cell lines (16.97 and 11.62, respectively, compared to 6.48). The docking scores also correlated well with the experimental activity and selectivity data. A comparison of the binding scores, average IC₅₀, and average selectivity of the most active compounds is presented in Table 4.

TABLE 4
Binding scores, average IC₅₀, and average
selectivity of the most active compounds.

| Compound No. | Binding score | Average IC₅₀ | Average selectivity |
|---|---|---|---|
| 6 | −8.467 | 1.214 | 33.34 |
| 13 | −7.952 | 2.046 | 18.5 |
| 19 | −7.062 | 1.803 | 4.02 |
| 20 | −6.889 | 1.676 | 2.92 |
| Doxorubicin | — | 1.462 | 22.5 |

Phthalazine compounds with substitutions at position 1 with various alkyl and aryl substituted nitrogen and nitrogen heterocycles are described, which enhanced both the affinity and selectivity towards the PARP target. The docking scores aligned with the experimental data, validating the compounds' interactions with the target and their selectivity. A new series of phthalazine derivatives have been produced by utilizing environmentally friendly processes that use microwave irradiation in high yield and short reaction times. The utilization of the dynamic microwave power system resulted in highly effective heating of the materials, leading to reduced reaction times and enhanced yields in the preparation of the target phthalazine derivatives. The synthesized phthalazine derivatives demonstrated broad anticancer activity against three different cell lines: MCF7, HCT116, and HepG2. Compound 6 was identified as the most active and selective compound, showing results comparable to doxorubicin. The docking results aligned with the experimental data and highlighted the influence of substituting position 1 of the phthalazine derivatives on their binding affinity to the PARP-1 active site. The computational analysis findings identified the phthalazine compounds as a novel scaffold for inhibiting PARP-1.

Numerous modifications and variations of the present disclosure are possible considering the above teachings. It is, therefore, to be understood that within the scope of the appended claims, the invention may be practiced otherwise than as specifically described herein.

1. A method of synthesizing a phthalazine compound by microwave irradiation, comprising:
reacting phosphorus oxychloride, phosphorus pentachloride, and 4-(4-isopropyl-phenyl)-2H-phthalazin-1-one in an organic solvent to obtain a reaction mixture comprising 1-chloro-4-(4-isopropyl-phenyl)-phthalazine;
mixing at least one selected from the group consisting of a hydrazine, an amine, an acetone, a dinitrile, a dicarbonyl and a semicarbazide with the reaction mixture obtain a final mixture, then microwave irradiating the final mixture at a power of 250 to 450 watt (W) for 5 to 30 minutes (min) to form the phthalazine compound.

2. The method of claim 1, wherein the phthalazine compound is at least one selected from the group consisting of 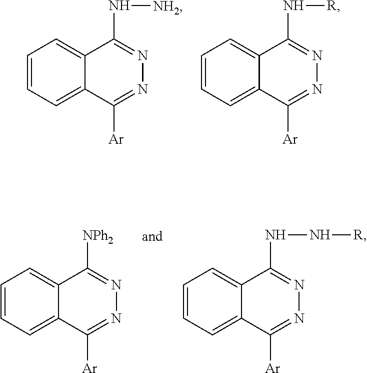
wherein
Ar is 4-isopropyl-phenyl, and
R is a —CH₃, —C₆H₅, —C₁₀H₇, 4-(Me)-C₆H₄, 4-(OMe)-C₆H₄, 3,4-(Me)₂-C₆H₃, 3-(NO₂)—C₆H₄, 2,4-(NO₂)₂—C₆H₃, or —(CO)-2-thiophene.

3. The method of claim 1, wherein the phthalazine compound is at least one selected from the group consisting of 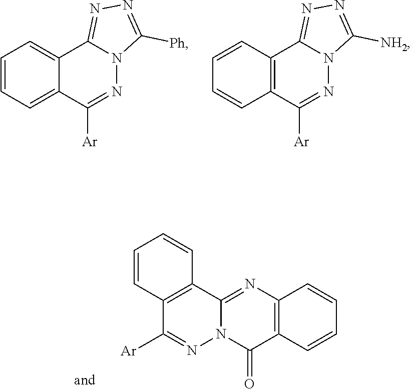
wherein Ar is 4-isopropyl-phenyl.

4. The method of claim 1, wherein the reaction mixture is mixed with at least one semicarbazide selected from the group consisting of semicarbazide hydrochloride, thiosemicarbazide, 4-phenylsemicarbazide, 4-methylsemicarbazide, 4-nitro semicarbazide, 1-acetylsemicarbazide, and 1-phenylsemicarbazide.

5. The method of claim 1, wherein the phthalazine compound is at least one selected from the group consisting of 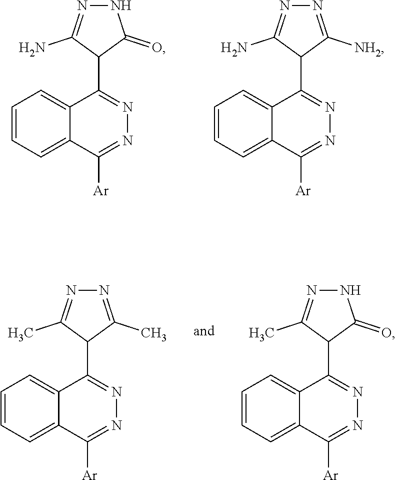
wherein Ar is 4-isopropyl-phenyl.

6. The method of claim 1, wherein the reaction mixture is mixed with at least one dicarbonyls selected from the group consisting of acetylacetone, ethylacetone, dimethyl malonate.

7. The method of claim 1, wherein the organic solvent is benzene.

8. The method of claim 1, wherein the microwave irradiating comprises microwave irradiating the final mixture at a power of 300 to 400 W for 9 to 20 min.

9. The method of claim 1, wherein the reaction mixture is mixed with at least one amine selected from the group including a methylamine, an aniline, a p-toluidine, a p-anisidine, a p-naphthylamine, an anthranilic acid, and a diphenylamine.

10. The method of claim 1, wherein the phthalazine compound is at least one selected from the group consisting of 4-(4-isopropyl-phenyl)-phthalazin-1-yl]-hydrazine, N-[4-(4-isopropyl-phenyl)-phthalazin-1-yl]-N′-phenyl-hydrazine, N-[4-(4-isopropyl-phenyl)-phthalazin-1-yl]-N′-(3,4-dimethyl phenyl-hydrazine, N-[4-(4-isopropyl-phenyl)-phthalazin-1-yl]-N′-(3-nitro-phenyl)-hydrazine, N-[4-(4-isopropyl-phenyl)-phthalazin-1-yl]-N′-(2,4-dinitro-phenyl)-hydrazine, N-[4-(4-isopropyl-phenyl)-phthalazin-1-yl]-hydrazide-N′-thiophene-2-carboxylic acid, 6-(4-isopropyl-phenyl)-3-phenyl-[1,2,4]tria-zolo[3,4-a]-phthalazine, 6-(4-isopropyl-phenyl)-[1,2,4]triazolo[3,4-a]-phthalazin-3-yl-amine, 4-(4-isopropyl-phenyl)-phthalazin-1-yl]-methyl-amine, 4-(4-isopropyl-phenyl)-phthalazin-1-yl]-phenyl-amine, 4-(4-isopropyl-phenyl)-phthalazin-1-yl]-p-tolyl-amine, 4-(4-isopropyl-phenyl)-phthalazin-1-yl]-(4-methoxy-phenyl)-amine, 4-(4-isopropyl-phenyl)-phthalazin-1-yl]-naphthalen-2-yl-amine, 5-(4-isopropyl-phenyl)-6,6a,12-triaza-ben-zo[a]anthracen-7-one, 4-(4-isopropyl-phenyl)-phthalazin-1-yl]-diphenyl-amine, 1-(3,5-Dimethyl-4H-pyrazol-4-(4-isopropyl-phenyl)-phthalazine, 4-[4-(4-isopropyl-phenyl)-phthalazin-1-yl]-5-methyl-2,4-dihydro-pyrazol-3-one, 5-amino-4-[4-(4-isopropyl-phenyl)-phthala-zin-1-yl]-2,4-dihydro-pyrazol-3-one, and 4-[4-(4-isopropyl-phenyl)-phthalazin-1-yl]-4H-pyrazole-3,5-diamine.

11. The method of claim 1, wherein the microwave irradiating comprises microwave irradiating the final mixture at a power of 300 to 400 W for 9 to 10 min.

12. The method of claim 1, wherein the hydrazine is at least one selected from the group consisting of a hydrazine hydrate, a phenylhydrazine, a 3,4-dimethylphenylhydrazine, a 3-nitrophenylhydrazine, a 2,4-dinitrophenyl hydrazine, a 2-thiophene hydrazine, and a benzoyl hydrazine.

13. A method of treating a cancer in a subject, comprising:
contacting the cancer with a 0.01 to 50 micromolar (μM) solution of the phthalazine compound of claim 1,
wherein the cancer is at least one cancer line selected from the group consisting of Michigan Cancer Foundation-7 (MCF7), Human Colon Tumor 116 (HCT116), and Human Hepatocellular Carcinoma G2 (HepG2).

14. The method of claim 13, wherein the cancer is MCF7, and
wherein the phthalazine compound has a half-maximal inhibitory concentration (IC₅₀) of 5.5 μM or less.

15. The method of claim 13, wherein the cancer is HCT116, and
wherein the phthalazine compound has an IC₅₀ of 30 μM or less.

16. The method of claim 13, wherein the cancer is HepG2, and
wherein the phthalazine compound has an IC₅₀ of 5 μM or less.

17. The method of claim 13, wherein the cancer is MCF7, and
wherein the phthalazine compound has a selectivity index (SI) of 1 to 42.

18. The method of claim 13, wherein the cancer is HCT116, and
wherein the phthalazine compound has an SI of 1 to 68.

19. The method of claim 13, wherein the cancer is HepG2, and
wherein the phthalazine compound has an SI of 1 to 17.

20. The method of claim 13, wherein the phthalazine compound has a binding score of −10 to −5.
